(19) United States
(12) Patent Application Publication
(10) Pub. No.: US 2023/0110223 A1
(43) Pub. Date: Apr. 13, 2023

(54) ECHOGENIC COMPOSITIONS AND METHODS OF USE THEREOF FOR THE TREATMENT OF PAIN

(71) Applicant: InSitu Biologics, Inc., Oakdale, MN (US)

(72) Inventors: William J. Taylor, Woodbury, MN (US); Kelsey Pflepsen, Minneapolis, MN (US); Mark Ereth, Oakdale, MN (US)

(21) Appl. No.: 17/965,738

(22) Filed: Oct. 13, 2022

Related U.S. Application Data
(60) Provisional application No. 63/255,335, filed on Oct. 13, 2021.

(51) Int. Cl. A61K 31/445; A61K 47/12; A61K 47/36; A61K 9/06; A61K 9/107

(52) U.S. Cl. CPC A61K 31/445; A61K 9/06; A61K 9/1075; A61K 47/12; A61K 47/36

(57) ABSTRACT

In general a hydrogel-lipid-microparticle drug delivery matrix that is tunable and has the ability to deliver sustained release pharmaceutical and / or active agents. The hydrogel-lipid-microparticle drug delivery matrix being echogenic.

### CROSS-REFERENCE TO RELATED APPLICATION(S

This application claims the benefit under 35 U.S.C. § 119(e) to U.S. Provisional Application 63/255,335, filed Oct. 13, 2021, and entitled Echogenic Compositions and Methods Of Use Thereof For The Treatment Of Pain, which is hereby incorporated herein by reference in its entirety for all purposes.

### TECHNICAL FIELD

The disclosure relates to pain management and treatment.

### BACKGROUND

Liposomal bupivacaine has been used in peripheral nerve blocks to prolong duration of action of the local anesthetic and to reduce post-operative pain and perioperative opioid use. In practice, that preparation often falls short of eliminating opiate use for surgical patients in many settings. The pharmacokinetic profile of the delivery system and migration of the local anesthetic in tissue jointly contribute to limited efficacy and duration of action at target sites.

Acute post-op pain is estimated to need at least 5-7 days of pain relief coverage. To that end, advancements in perioperative medicine endeavor to eliminate opioids from a post-operative treatment regimen, but have fallen short to date. Numerous opioid-limiting techniques such as intrathecal morphine, medicinal adjuncts, and regional anesthetics of standard and extended release preparations demonstrate limited effectiveness to 24-48 hours of effective analgesia—often requiring narcotics to supplement waning analgesia.

There is a need in the art for compositions and methods that are effective in providing sustained post-operative pain relief without the use of opiate based medications.

### BRIEF SUMMARY

Disclosed herein are echogenic compositions for the delivery of an API (e.g. an anesthetic agent) to a subject and methods of use thereof. A method for treating pain in a subject is provided comprising administering an echogenic composition to a target site in a subject and confirming delivery of the echogenic composition to the target site with ultrasound. In certain embodiments the echogenic composition comprises an aqueous carrier and a plurality of lipid microparticles dispersed within the aqueous carrier, wherein the plurality of lipid microparticles comprise an anesthetic agent. In certain embodiments, the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%. In certain embodiments, the volumetric ratio between the aqueous carrier and the lipid microparticles is from about 70-80 the aqueous carrier to about 30-20 lipid microparticles.

According to further embodiments, the plurality of lipid microparticles are comprised of a paraffin, a triglyceride, and/or a wax. In certain implementations, the lipid microparticle is a wax and the wax is a carnauba wax. In further embodiments, the lipid microparticles comprise a wax or a mixture of a wax and a fatty acid, wherein the fatty acid is C4 or greater. In yet further embodiments, the plurality of lipid microparticles comprise stearic acid and tributyrate. In exemplary implementations, the stearic acid and tributyrate are present at a ratio of from about 0.1% to about 30%.

In certain implementations, the anesthetic agent is present within the lipid microparticle in a crystalline form.

Further disclosed herein is echogenic composition comprising an aqueous carrier and a lipid phase dispersed into droplets within the aqueous carrier, and an undissolved crystalline anesthetic agent within the lipid phase. In certain implementations, the lipid phase is a triglyceride. In exemplary implementations, the triglyceride is a liquid at 25° C.

In certain embodiments, the lipid phase droplets are from about 500 nm to about 100 µm in diameter. In further embodiments, the lipid phase droplets are from about 500 nm to about 5 µm in diameter.

In certain embodiments, the aqueous phase comprises hyaluronic acid. In further embodiments, the hyaluronic acid present in an amount from about 0.1% to about 1%.

Further disclosed herein is an echogenic composition for the treatment of pain comprising: a continuous aqueous phase comprising an emulsifier and a polyol; a lipid phase comprising a triglyceride, wherein the triglyceride is liquid at 25° C. and wherein an undissolved crystalline anesthetic agent within the triglyceride; and wherein the lipid phase is emulsified within the continuous aqueous phase.

According to certain embodiments, the emulsifier is hyaluronic acid. In exemplary implementations of these embodiments, the hyaluronic acid is present in an amount from about 0.15% to about 1%.

According to further embodiments, the lipid phase further comprises a phospholipid. In certain implementations, the phospholipid is present in an amount from about 0.1% to about 2.0% of the lipid phase.

In certain embodiments, the lipid phase further comprises an antioxidant. In certain implementations, the antioxidant is present in an amount of from about 0.01% to about 1% (w/v) of the composition. Exemplary antioxidants include any suitable lipophilic antioxidant. In certain implementations, the antioxidant is a tocopherol (e.g. alpha tocopherol).

In certain embodiments, the lipid phase is from about 10% to about 40% (w/v) of the composition.

In further embodiments, the polyol is glycerol. In exemplary implementations, glycerol is present in an amount of from about 0.25 to about 2.25% (w/v) of the composition.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is an image from an ultrasound showing an echogenic needle pre-injection, according to certain embodiments.

FIG. 1B is an image from an ultrasound showing injection of an INSB200 (hydrogel-lipid-microparticle ropivacaine matrix) injection demonstrating dense echogenicity with echo-dense shadow, according to certain embodiments.

FIG. 2A is an image from an ultrasound showing an echogenic needle pre-injection, according to certain embodiments.

FIG. 2B is an image from an ultrasound showing injection of an INSB200 (hydrogel-lipid-microparticle ropivacaine matrix) injection demonstrating dense echogenicity, according to certain embodiments.

FIG. 3 shows a shallow injection of a control article into a pig muscle; the needle can be seen in the right side of the image field of view.

FIG. 4 shows a pig muscle post shallow injection of the control test article (1% hyaluronic acid solution); the needle is present in right side of image field of view.

FIG. 5 shows a deep injection of a control article into a pig muscle; the needle can be seen in the right side of the image field of view.

FIG. 6 shows a pig muscle post deep injection of the control test article; the needle is shown in the top right image field of view.

FIG. 7 shows a pig muscle prior to shallow injection of the 0.5% lipid microparticle formulation; the needle is in the right image field of view.

FIG. 8 shows a pig muscle post shallow injection of the 0.5% lipid microparticle formulation; the needle is present in the right image field of view.

FIG. 9 shows a pig muscle prior to deep injection of the 0.5% lipid microparticle formulation; the needle is present in the right image field of view.

FIG. 10 shows a pig muscle post deep injection of the 0.5% lipid microparticle formulation; the needle is present in the right image field of view.

FIG. 11 shows a pig muscle prior to shallow injection of the 1% lipid microparticle formulation; the needle is visible in the image right field of view.

FIG. 12 shows a pig muscle post shallow injection of the 1% lipid microparticle formulation; the needle is visible in the image right field of view.

FIG. 13 shows a pig muscle prior to deep injection of the 1% microparticle formulation; the needle is shown in image right side field of view.

FIG. 14 shows a pig muscle post deep injection of the 1% microparticle formulation; the needle is shown in image right side field of view.

FIG. 15 shows a pig muscle prior to shallow injection of the 10% lipid microparticle formulation; the needle is present in the right side field of view.

FIG. 16 shows a pig muscle post shallow injection of the 10% lipid microparticle formulation.

FIG. 17 shows a pig muscle prior to deep injection of the 10% lipid microparticle formulation; the needle is present in the right side image field of view.

FIG. 18 shows a pig muscle post deep injection of the 10% lipid microparticle; the needle is present in the right side field of view.

FIG. 19 shows a pig muscle prior to shallow injection of the 30% lipid microparticle formulation; the needle is present in the right side image field of view.

FIG. 20 shows a pig muscle post shallow injection of the 30% lipid microparticle formulation; the needle is present in the top right side of image field of view.

FIG. 21 shows a pig muscle prior to deep injection of the 30% lipid microparticle formulation; the needle is present in the top right field of view.

FIG. 22 shows a pig muscle post deep injection of the 30% microparticle formulation; the needle is present in the top right quadrant of image field of view.

FIG. 23 shows a pig muscle prior to shallow injection of the 10% lipid homogenized formulation; the needle is seen in the right side of the image field of view.

FIG. 24 shows a pig muscle post shallow injection of the 10% lipid microparticle formulation containing 13% ropivacaine; the needle can be seen in the right side field of view.

FIG. 25 shows a pig muscle prior to deep injection of the 10% homogenized lipid microparticle formulation containing 13% ropivacaine; the needle is shown in the right side image field of view.

FIG. 26 shows a pig muscle post deep injection of the 10% homogenized lipid microparticle formulation containing 13% ropivacaine; the needle is seen in the right side image field of view.

FIG. 27 shows a pig muscle prior to shallow injection of the 30% lipid microparticle formulation containing stearic acid and tributyrate; the needle is seen in the right side image field of view.

FIG. 28 shows a pig muscle post shallow injection of the 30% lipid microparticle formulation containing stearic acid and tributyrate; the needle is seen on far right of image field of view.

FIG. 29 shows a pig muscle prior to deep injection of the 30% microparticle formulation containing stearic acid and tributyrate; the needle is shown as a diagonal line starting in top right of image field of view.

FIG. 30 shows a pig muscle post deep injection of the 30% lipid microparticle formulation containing stearic acid and tributyrate; the needle is shown as diagonal line starting in top right quadrant of image and entering test article cloud.

FIG. 31 shows a pig muscle prior to shallow injection of the 10% stearic acid and tributyrate microparticle formulation; the needle is seen entering from the right side of the image field of view.

FIG. 32 shows a pig muscle post shallow injection of the 10% stearic acid and tributyrate microparticle formulation; the needle is seen entering the right side of the image of field of view.

FIG. 33 shows a pig muscle prior to deep injection of 10% lipid microparticle formulation containing stearic acid and tributyrate; the needle is seen on the right side image field of view.

FIG. 34 shows a pig muscle post deep injection of the 10% lipid microparticle formulation containing stearic acid and tributyrate.

FIG. 35 shows a pig muscle prior to shallow injection of the 10% caprylic acid and tristearate formulation; the needle is seen in the right side field of view.

FIG. 36 shows a pig muscle post shallow injection of the 10% caprylic acid and tristearate formulation; the needle is seen in the right side field of view.

FIG. 37 shows the sciatic nerve in the middle of the field of view, where no drug product has been injected (Dose A Pig 1 baseline).

FIG. 38 shows the drug product completely engulfing the sciatic nerve and shown to be within the intrafascial space below the bright line midway in the field of view shows a pig muscle (Dose A Pig 1 Complete injection with drug product).

FIG. 39 shows the sciatic nerve in the middle of the field of view, where no drug product has been injected (Dose A Pig 2).

FIG. 40 shows emulsion drug product at the end of the injection procedure. The drug product appears as a cloud that has enveloped the sciatic nerve.

FIG. 41 shows injection of the emulsion drug product mid injection. The drug product cloud has begun to spread out in the fascial plane.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions may be available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991); March’s Advanced Organic Chemistry, (John Wiley and Sons, 4th Edition); and Larock’s Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, “echogenic composition” means a composition that gives rise to reflections of ultrasound waves and is thus detectable using standard ultrasound imaging techniques.

As used herein, the terms “treat,” and “prevent” as well as words stemming therefrom, do not necessarily imply 100% or complete treatment or prevention. Rather, there are varying degrees of treatment or prevention of which one of ordinary skill in the art recognizes as having a potential benefit or therapeutic effect. In this respect, the methods of the present invention can provide any amount of any level of treatment or prevention of a disease or medical condition in a mammal. Furthermore, the treatment or prevention provided by the method can include treatment or prevention of one or more conditions or symptoms of the disease or medical condition. For example, with regard to methods of treating pain, the method in some embodiments, achieves a diminution in or elimination of pain in a subject. Also, for purposes herein, “prevention” can encompass delaying the onset of the disease, or a symptom or condition thereof. The term “treating” includes prophylaxis of the specific disorder or condition, or alleviation of the symptoms associated with a specific disorder or condition and/or preventing or eliminating said symptoms. For example, as used herein the term “post-operative pain” refers in general to producing a diminution or alleviation of pain associated with recovering from a surgical procedure.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman’s The Pharmaceutical Basis of Therapeutics, Chapter 1, pp. 1-46, latest edition, Pergamagon Press, which is hereby incorporated by reference in its entirety, and the references cited therein.

Disclosed herein are echogenic compositions for providing controlled and/or sustained release of an active pharmaceutical ingredient (API) in the body of a subject.

Disclosed herein are echogenic compositions for the local delivery of an API (e.g. an anesthetic agent) to a subject and methods of use thereof. A method for treating pain in a subject is provided comprising administering an echogenic composition to a target site in a subject and confirming delivery of the echogenic composition to the target site with ultrasound.

In certain embodiments the echogenic composition comprises an aqueous carrier and a plurality of lipid microparticles dispersed within the aqueous carrier, wherein the plurality of lipid microparticles comprise an API (e.g., anesthetic agent). In certain embodiments, the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%. In certain embodiments, the volumetric ratio between the aqueous carrier and the lipid microparticles is from about 70-80 the aqueous carrier to about 30-20 lipid microparticles.

According to further embodiments, the plurality of lipid microparticles are comprised of a paraffin, a triglyceride, and/or a wax. In certain implementations, the lipid microparticle is a wax and the wax is a carnauba wax. In further embodiments, the lipid microparticles comprise a wax or a mixture of a wax and a fatty acid, wherein the fatty acid is C4 or greater. In yet further embodiments, the plurality of lipid microparticles comprise stearic acid and tributyrate. In exemplary implementations, the stearic acid and tributyrate are present at a ratio of from about 0.1% to about 30%. Other possible composition of the lipid microparticle and/or lipid phase are described further below.

In certain implementations, the anesthetic agent is present within the lipid microparticle in a crystalline form. In further implementations, the anesthetic agent is dissolved within the lipid microparticle

Further disclosed herein is echogenic composition comprising an aqueous carrier and a lipid phase dispersed into droplets within the aqueous carrier, and an undissolved crystalline anesthetic agent within the lipid phase. In certain implementations, the lipid phase is a triglyceride. In exemplary implementations, the triglyceride is a liquid at 25° C. As will be appreciated by those skilled in the art, in contrast to the echogenic composition described above, in these embodiments, the lipid phase is not in the form of solid microparticles but in the form of liquid microdroplets and/or emulsified within the aqueous carrier phase.

In certain embodiments, the lipid phase droplets are from about 500 nm to about 100 µm in diameter. In further embodiments, the lipid phase droplets are from about 500 nm to about 5 µm in diameter. In certain embodiments, lipid phase droplets formed around solid crystalized API are less than about 5 µm.

In certain embodiments, the aqueous phase comprises hyaluronic acid. In further embodiments, the hyaluronic acid present in an amount from about 0.1% to about 1%. The HA in these embodiments may or may not be cross linked to form a hydrogel. In embodiments where no hydrogel is formed, the HA may be unsubstituted (e.g. lack tyramine substitution as described below).

Further disclosed herein is an echogenic composition for the treatment of pain comprising a continuous aqueous phase comprising an emulsifier and a polyol; a lipid phase comprising a triglyceride, wherein the triglyceride is liquid at 25° C. and wherein an undissolved crystalline anesthetic agent within the triglyceride; and wherein the lipid phase is emulsified within the continuous aqueous phase.

According to certain embodiments, the emulsifier is hyaluronic acid. In exemplary implementations of these embodiments, the hyaluronic acid is present in an amount from about 0.15% to about 1%. According to further embodiments, suitable emulsifiers include, but are not limited to phospholipids, and poly sorbates (tween surfactant). In further embodiments, fatty acid (0.1-2%) can function as emulsifier.

According to further embodiments, the lipid phase further comprises a phospholipid. Phospholipids serve as an additional emulsifier and provide for a stabilized emulsified echogenic composition. In certain implementations, the phospholipid is present in an amount from about 0.1% to about 2.0% of the lipid phase. Exemplary phospholipids include but are not limited to lecithins. Suitable lecithins include but are not limited to plant based lecithins such as soybean lecithin, corn germ oil lecithin, rapeseed lecithin including lecithin derived from canola, field mustard and other rape seed variants and hybrids, rice oil lecithin, sunflower lecithin, cotton seed lecithin, peanut lecithin, palm oil lecithin, marine oil lecithin, biomass lecithin, and mixtures thereof. Also suitable are certain animal based lecithins, including but not limited to egg yolk lecithin, and/or milk lecithin and or mixtures thereof.

In certain embodiments, the lipid phase further comprises an antioxidant. In certain implementations, the antioxidant is present in an amount of from about 0.01% to about 1% (w/v) of the composition. Exemplary antioxidants include any suitable lipophilic antioxidant. In certain implementations, the antioxidant is a tocopherol (e.g. alpha tocopherol) Other lipophilic antioxidants (e.g. lycopene and beta carotene) are also suitable.

In certain embodiments, the lipid phase is from about 10% to about 40% (w/v) of the composition.

In further embodiments, the polyol is glycerol. In exemplary implementations, glycerol is present in an amount of from about 0.25 to about 2.25% (w/v) of the composition.

In certain embodiments, the compositions and methods herein may eliminate the need for perioperative opioid use in select patients.

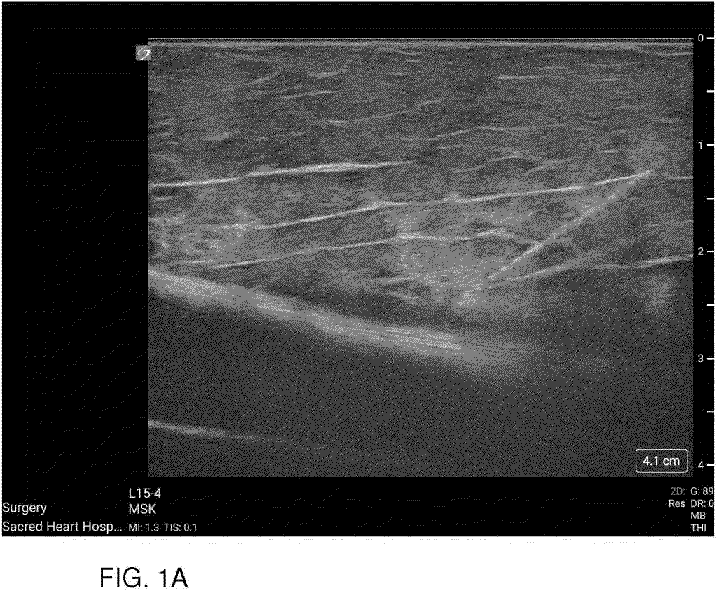

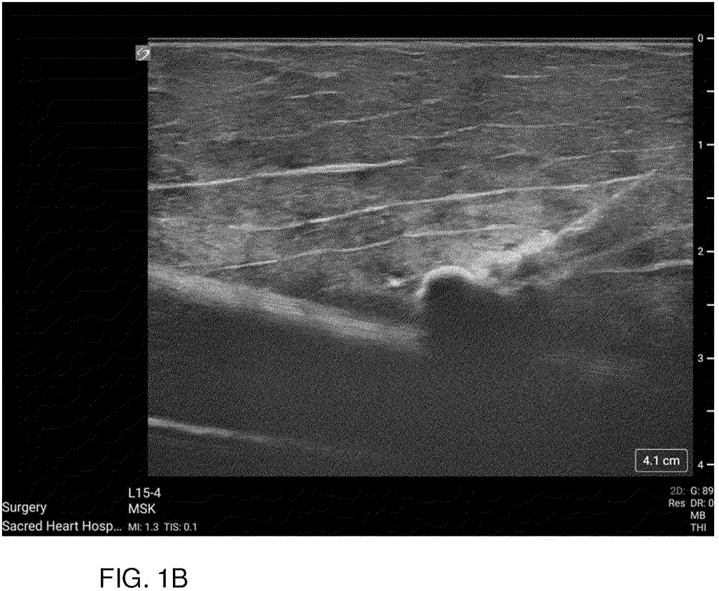

The disclosed echogenic compositions have unique characteristics over known anesthetic preparations. In various embodiments, the disclosed echogenic compositions demonstrate echogenicity with injection. In further embodiments, the disclosed echogenic compositions demonstrate no significant tissue spread once delivered to the target site when compared to other aqueous local anesthetic preparations. The ultrasound characteristics of aqueous local anesthetics show a non-echogenic deposit of medicant with significant tissue migration. Injection of the disclosed echogenic compositions results in an echogenic pocket of medicant that remains at the site of injection without appreciable tissue spread (FIGS. 1A and 1B).

These two properties of the disclosed echogenic compositions are distinct from other agents and provide clinical benefits. First, the disclosed echogenic compositions allow for precise, non-migrating administration of a local anesthetic drug matrix which, in these embodiments, may benefit in certain non-planar peripheral nerve blocks (such as sciatic, femoral, and brachial plexus) which in the case of the upper extremity could reduce the incidence of phrenic nerve blockade. In further embodiments, localized, non-migrating anesthetic delivery may allow for more precise and prolonged analgesia with lower drug volumes.

In further embodiments, the disclosed echogenic compositions have echogenic properties that aid in confirming placement of medication during regional anesthetic techniques. This is true with deeper ultrasound-guided injections, where aqueous formulations are often poorly visualized.

In certain embodiments, disclosed echogenic compositions enables clinicians to be more precise when depositing local anesthetic and positively identify placement of medicant for procedural confirmation. In various embodiments, effectually, medication remains where it was placed—utilizing less medication with a pain relieving effect of up to 6 days or more.

In still further embodiments, the disclosed echogenic compositions may be used to deliver other medications under ultrasound guidance (anti-inflammatory, chemotherapeutics, and antibiotics). In such cases, the echogenic property would aid in precise medication administration for proceduralists and anesthesiologists looking to positively identify the site of injection with a high degree of sensitivity.

According to certain implementations, the disclosed echogenic composition comprises a aqueous phase (also sometimes referred to as “carrier phase”) and a lipid phase that contains an active pharmaceutical ingredient (API) that is released to a biological system over a targeted treatment duration and allows for verification of proper target placement through ultrasound imaging. In this context the primary function of the carrier phase is to disperse the drug reservoir particles (drug carrying component) to create a stable homogenous mass and allow the use of delivery devices, such as a syringe, to draw up a dose from a container and deliver it to a target tissue, i.e. parenteral injection, intravascular injection, wound instillation, wound packing or mass formation or coating on a tissue surface, while providing the ability to verify the proper targeting of the delivery through ultrasound imaging of the echogenic signal of the composition. The drug reservoir/lipid phase is a separate physical phase, a collection of particles in some embodiments, that are contained within the carrier phase but not indistinguishable from the carrier phase. The lipid phase contains the active pharmaceutical agent dissolved in the lipid material and may be in an unsaturated, saturated, super saturated, or saturated with pure pharmaceutical phase material (crystals for small molecules) state. In some forms the carrier may also contain the contain the API in a different form than the reservoir, such as an API salt in an aqueous carrier and the base form API in a lipid phase. The system is not set to be only aqueous/hydrophobic, but can be opposite, or separate physical phase (polymer).

In certain embodiments, the aqueous phase is a hydrogel. The term “hydrogel” as used herein refers to a three-dimensional, hydrophilic or amphiphilic polymeric network capable of taking up large quantities of water. The networks are composed of homopolymers or copolymers (referred to at times herein as a polymer backbone) and are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allow them to swell in aqueous media.

In certain implementations, the hydrogel is comprised of tyramine substituted hyaluronic acid (THA) which is cross linked through di-tyramine linkages. Preparation of THA is described U.S. Pat. No. 6,982,298, which is incorporated herein by reference in its entirety. The degree of tyramine substitution has a significant impact on the properties of the resulting hydrogel. Throughout the instant disclosure, degree of tyramine substitution refers to the percentage of all HA carboxyl groups that have been substituted by tyramine. For example, in a 2% substituted THA, 2% of all HA carboxyl groups have been substituted by tyramine. The percent tyramine substitution within each THA preparation is calculated by measuring: 1) the concentration of tyramine present in the preparation, which is quantitated spectrophotometrically based on the unique UV-absorbance properties of tyramine at 275 nm; and 2) the concentration of total carboxyl groups in the HA preparation, which is quantitated spectrophotometrically by a standard hexuronic acid assay.

As described further below, hydrogel can be tuned to possess a specific osmolality, physical property, API elution rate or tissue response by adjusting the concentration of the tyramine substituted polymer backbone, the degree of substitution of the tyramine on the polymer backbone, the molecular weight of polymer backbone, the hydrophilicity of the polymer backbone, the type of polymer backbone and concentration of target molecules, salts, buffers or drug depot (reservoir) particles contained within the hydrogel.

The hydrogel physical properties can be adjusted by changing the concentration of tyramine substituted polymer backbone. In certain embodiments, liquid-like hydrogels are created by keeping the tyramine substituted polymer backbone less than 0.35% of the aqueous carrier phase for a 1.5% substituted gel. Liquid-like hydrogels are more appropriate for intravascular injection, intrathecal injection or other tissue sites that cannot tolerate occlusion or blocking vessels or tissue structures. Dense hydrogel particles can be formed by increasing tyramine substitution on the polymer backbone. 5% or higher degrees of substitution will form solid like hydrogel particles at low concentrations and very dense particles at 7% or higher concentrations. Dense particles are more appropriate for instillation into wound sites. In certain implementations, dense hydrogel particles are used deliver biological molecules and polar APIs. In contrast, in implementations where the API is hydrophobic, lipid microparticles are suitable.

The hydrogel physical property can also be adjusted by changing the type of polymer backbone. For example, collagen can be used as a polymer backbone, and it is much less hydrophilic than a saccharide-based polymer backbone. The collagen gels do not swell in the same way that polysaccharide gels and have much lower molecular weights and concentrations. It can be envisioned that the polymer backbone can be changed to take advantage of a single polymers physical & chemical characteristics, or several species can be combined in a copolymer or block copolymer in a way that will change the gel physical and chemical properties, the way in which the body interacts with the gel. Some polymers will have a higher affinity rate for an API and API elution rates will be impacted if a polymer or section of polymer has been chosen that has a higher binding affinity for the API. It is also envisioned that by using polymer/API combinations in which binding affinity of the API to the backbone polymer is pH or temperature dependent, the gel formulation can be adjusted to maximize binding at T=0 and then releasing more API as the pH and temperature approaches physiological conditions after exposure to target tissues. In further implementations, API diffusion rate is affected by changing the melting point of the lipid microparticles (described further below) as enhanced diffusion can be reached as the liquid-liquid interface (achieved upon melting of the lipid microparticle) diffusion flux is higher than solid to liquid interface.

The hydrogel osmolality can also be tuned by the degree of tyramine substitution, and concentration. Concentrated highly substituted hydrogels by themselves will expel water or undergo syneresis, but by increasing the concentration of the polymer backbone in the example of hyaluronic acid, or adding salts, buffers and/or API materials to the formulation the gel can be made to be osmotically neutral or swell slightly. For example, a 5.5% substituted gel can be created that will swell if the backbone polymer concentration is set to 1.5%. It is envisioned that a gel can be created to swell even more as the osmolality of the gel is increased by adding buffers, salts and API ingredients. In certain aspects, the hydrogel is comprised of tyramine substituted hyaluronic acid. According to certain implementations, the hydrogel is formed through di-tyramine crosslinking.

The advantage of controlling backbone polymer concentration and degree of substitution can also be used to elicit a biological response. Using a gel with 1.5% substitution and 0.5% concentration will absorb fluids from the tissues surrounding the hydrogel implant. The capillary beds will constrict and in some cases such as a traumatic wound site, will decrease or stop bleeding from a wounded surface. The reduction of blood flow in tissues near the implant will also slow removal of an eluting API from the implant site. Tissues which may be harmed from reduced perfusion such as cartilage or joint spaces can have a hydrogel tuned to be osmotically neutral to prevent negative impacts due to reduced perfusion. In clinical applications where it is desirable to stop bleeding from a highly vascular bleeding surface such as the liver, a very concentrated hydrogel that appear to be dry or almost dry will absorb sera and exudate very quickly and dehydrate the wound site. In the case where procoagulants such as fibrin, tranexamic acid, aminocaproic acid or fibrin, etc. the hydrogel can promote coagulation at the wound site via two pathways, capillary bed constriction and blood coagulation.

Hydrogel density can be used to control rate of elution of an API from the gel to the target tissue. A 1% hydrogel will elute API for >72 hrs, but a 10% gel will extend elution time to over 100 hrs. Depending on API or biologic material size and affinity for the aqueous phase components, the elution rate can be tuned to a desired elution rate that will allow the hydrogel to act as a drug reservoir for several days.

In certain aspects, the degree of tyramine substitution of hyaluronic acid hydroxyl groups ranges from about 0.25% to about 8%. In further aspects, the degree of tyramine substitution of hyaluronic acid hydroxyl groups is about .5% to about 3%.

In still further aspects, the tyramine substituted hyaluronic acid is present in the aqueous phase at from about 0.1% to about 4%.

In certain implementations, the tyramine substituted hyaluronic acid is present in the aqueous phase from about 0.1 to about 1%. In further implementations, the tyramine substituted hyaluronic acid is present in the aqueous phase at about 0.25%.

In certain implementations the carrier phase is an aqueous carrier phase that is not a hydrogel. In certain implementations of these embodiments, the aqueous carrier phase comprises hyaluronic acid (e.g. from 0.15-1%) that is not tyramine substituted and does not otherwise provide for cross-linking and hydrogel formation of the aqueous phase. In these embodiments, the HA may serve as an emulsifier to facilitate stable emulsion of the lipid and aqueous carrier phases.

### Lipid Phase

In certain embodiments, the lipid phase is present in the form of lipid microparticles dispersed within the aqueous phase. In further embodiments, the lipid phase is liquid at room temperature/body temperature and the microdroplets a dispersed throughout the aqueous phase or emulsified into the aqueous phase.

According to certain embodiments, lipid phase of the disclosed echogenic composition is comprised of one or more fatty acids. In certain implementations, the one or more fatty acids have an even number of carbons. In certain implementations, the fatty acids are chosen from: stearic acid, oleic acid, myristic acid, caprylic acid, capric acid, lauric acid, palmitic acid, arachidic acid, lignoceric acid, cerotic acid, and mixtures of the forgoing.

In certain exemplary implementation, where the fatty acid microparticles are comprised of mixtures of fatty acids, the fatty acids are present at specific ratios. For example, in certain implementations, the mixture of fatty acids comprises a 90:10 ratio of steric to oleic acid.

Fatty acids of various carbon lengths are common throughout the living world and are utilized by animals as part of the cell membrane, as energy storage and for thermal regulation. Fatty acids are comprised of carboxylic acid attached to an aliphatic carbon chain. In general, they are insoluble in water but as the carbon chain length shortens, their acidity increases. Fatty acids can be saturated or contain no carbon-carbon double bonds. Or they may be unsaturated, containing one or more carbon-carbon double bonds in the aliphatic carbon chain. Mammalian organisms can process and create fatty acids with even numbered carbon chains. Odd numbered fatty acids are produced by some bacteria and are found in the milk of ruminants, but in most cases, they are even numbered due to the metabolic process that adds two carbons at a time to the chain. Table 1 lists fatty acids typically found in plant and animals. The lipid number lists the number of carbons in the aliphatic chain followed by the number of double bonds. In some listings, the location of the double bond is included with the lipid number. In most cases the fatty acids are usually part of a triglyceride molecule that may contain up to three fatty acids of the same or differing carbon lengths.

In certain implementations, even numbered carbon fatty acids are selected. Mixtures of fatty acids can be made to adjust the melting point of the microparticles. In certain implementations, a mixture of 90% stearic acid with 10% oleic acid is used. This creates a microparticle that melts at 95° F. A similar melting point is achieved by mixing 12% myristic acid 32% palmitic acid, 10% stearic acid, and 10% oleic acid. According to further embodiments, the FA microparticle is formed from a mixture of lauric acid, caprylic acid, and caproic acid. The key factors in choosing a microparticle formulation are melting point and API solubility in main component fatty acid. The melting point is important in that particles close to physiological body temperature will be a liquid or soft semi-solid which will increase diffusion rate across a liquid - liquid interface. This may be desirable or not desirable depending on the specific application. In certain embodiments, a combination of low melting point and high melting microparticles (e.g. below and above 37C) are combined. API solubility will change due to fatty acid chain length and microparticle formulation and it may be desirable to adjust API concentration and affinity for the main microparticle fatty acid component. In some formulations increasing molecular weight and chain length of the fatty acid will change solubility of a partially polar API counterintuitively. In certain embodiments, the concentration of anesthetic agent within a FA microparticle is from about 1-25% by weight.

According to certain alternative embodiments, odd numbered fatty acids are used as an alternative fatty acid in the formulations. Monounsaturated fatty acids such as oleic acid may be used as well alone or in combination with other fatty acids. In certain implementations, poly unsaturated fatty acids can be used, but are not preferable as they oxidize easily and depending on the formulation may polymerize. Monounsaturated fatty acids that are in a cis configuration (most plant sourced) are preferable.

According to certain alternative embodiments, the lipid microparticles comprise one or more triglyceride or a mixture of triglycerides the lipid microparticles comprise one or more triglyceride or a mixture of triglycerides. In further alternative embodiments, the lipid microparticle comprises a paraffin and/or a wax.

TABLE 1

List of Fatty Acids and Corresponding Lipid Numbers.

| | | | |
|---|---|---|---|
| Common Name | Chemical Name | Structural Formula | Lipid Numbers |
| Propionic acid | Propanoic acid | CH₃CH₂COOH | C3:0 |
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)sCOOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Carboceric acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Hentriacontylic acid | Hentriacontanoic acid | CH₃(CH₂)₂₉COOH | C31:0 |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic acid | CH₃(CH₂)₃₂COOH | C34:0 |
| Ceroplastic acid | Pentatriacontanoic acid | CH₃(CH₂)₃₃COOH | C35:0 |
| Hexatriacontylic acid | Hexatriacontanoic acid | CH₃(CH₂)₃₄COOH | C36:0 |
| Heptatriacontylic acid | Heptatriacontanoic acid | CH₃(CH₂)₃₅COOH | C37:0 |
| Octatriacontylic acid | Octatriacontanoic acid | CH₃(CH₂)₃₆COOH | C38:0 |
| Nonatriacontylic acid | Nonatriacontanoic acid | CH₃(CH₂)₃₇COOH | C39:0 |
| Tetracontylic acid | Tetracontanoic acid | CH₃(CH₂)₃₈COOH | C40:0 |

TABLE 2

Monounsaturated Fatty Acids

| Common Name | Chemical Name | Molecular Formula | Lipid Numbers C-Atoms: Double Bonds |
|---|---|---|---|
| Undecylenic | cis-10-undecenoic acid | C₁₀H₁₉COOH | 11:1 |
| Myristoleic | cis-9-tetradecenoic acid | C₁₃H₂₅COOH | 14:1 |
| Palmitoleic | cis-9-hexadecenoic acid | C₁₅H₂₉COOH | 16:1 |
| Palmitelaidic | trans-9-hexadecenoic acid | C₁₅H₂₉COOH | 16:1 |
| Petroselinic | cis-6-octadecenoic acid | C₁₇H₃₃COOH | 18:1 |
| Oleic | cis-9-octadecenoic acid | C₁₇H₃₃COOH | 18:1 |
| Elaidic | trans-9-octadecenoic acid | C₁₇H₃₃COOH | 18:1 |
| Vaccenic | cis-11-octadecenoic acid | C₁₇H₃₃COOH | 18:1 |
| Gondoleic | cis-9-eicosenoic acid | C₁₉H₃₇COOH | 20:1 |
| Gondolic | cis-11-eicosenoic acid | C₁₉H₃₇COOH | 20:1 |
| Cetoleic | cis-11-docosenoic acid | C₂₁H₄₁COOH | 22:1 |
| Erucic | cis-13-docosenoic acid | C₂₁H₄₁COOH | 22:1 |
| Nervonic | cis-15-tetracosaenoic acid | C₂₃H45COOH | 24:1 |

In certain embodiments, polyunsaturated fatty acids are used to create the microparticles either alone or in mixtures of other fatty acids. Polyunsaturated fats typically have a lower melting point than do their equivalent carbon number saturated fatty acid analogues. Examples of two essential fatty acids are Linoleic acid (C18:2) and α-Linoleic acid (C18:3). The human body cannot make these fatty acids but requires them and must obtain them through dietary intake. The body can metabolize them so they can be used to generate microparticle drug reservoirs, but they have multiple double bonds which oxidize easily and may react with some APIs.

TABLE 3

Omesa-3 Fatty Acids

| | | |
|---|---|---|
| Common name | Chemical name | Lipid Numbers |
| Hexadecatrienoic acid (HTA) | all-cis 7,10,13-hexadecatrienoic acid | 16:3 (n-3) |
| Alpha-linolenic acid (ALA) | all-cis-9,12,15-octadecatrienoic acid | 18:3 (n-3) |
| Stearidonic acid (SDA) | all-cis-6,9,12,15,-octadecatetraenoic acid | 18:4 (n-3) |
| Eicosatrienoic acid (ETE) | all-cis-11,14,17-eicosatrienoic acid | 20:3 (n-3) |
| Eicosatetraenoic acid (ETA) | all-cis-8,11,14,17-eicosatetraenoic acid | 20:4 (n-3) |
| Eicosapentaenoic acid (EPA, Timnodonic acid) | all-cis-5,8,11,14,17-eicosapentaenoic acid | 20:5 (n-3) |
| Heneicosapentaenoic acid (HPA) | all-cis-6,9,12,15,18-heneicosapentaenoic acid | 21:5 (n-3) |
| Docosapentaenoic acid (DPA, Clupanodonic acid) | all-cis-7,10,13,16,19-docosapentaenoic acid | 22:5 (n-3) |
| Docosahexaenoic acid (DHA, Cervonic acid) | all-cis-4,7,10,13,16,19-docosahexaenoic acid | 22:6 (n-3) |
| Tetracosapentaenoic acid | all-cis-9,12,15,18,21-tetracosapentaenoic acid | 24:5 (n-3) |
| Tetracosahexaenoic acid (Nisinic acid) | all-cis-6,9,12,15,18,21-tetracosahexaenoic acid | 24:6 (n-3) |

TABLE 4

Omega 6 Fatty Acids

| | | |
|---|---|---|
| Common name | Chemical name | Lipid Numbers |
| Linoleic acid (LA) | all-cis-9,12-octadecadienoic acid | 18:2 (n-6) |
| Gamma-linolenic acid (GLA) | all-cis-6,9,12-octadecatrienoic acid | 18:3 (n-6) |
| Eicosadienoic acid | all-cis-11,14-eicosadienoic acid | 20:2 (n-6) |
| Dihomo-gamma-linolenic acid (DGLA) | all-cis-8,11,14-eicosatrienoic acid | 20:3 (n-6) |
| Arachidonic acid (AA) | all-cis-5,8,11,14-eicosatetraenoic acid | 20:4 (n-6) |
| Docosadienoic acid | all-cis-13,16-docosadienoic acid | 22:2 (n-6) |
| Adrenic acid (AdA) | all-cis-7,10,13,16-docosatetraenoic acid | 22:4 (n-6) |
| Docosapentaenoic acid (Osbond acid) | all-cis-4,7,10,13,16-docosapentaenoic acid | 22:5 (n-6) |
| Tetracosatetraenoic acid | all-cis-9,12,15,18-tetracosatetraenoic acid | 24:4 (n-6) |
| Tetracosapentaenoic acid | all-cis-6,9,12,15,18-tetracosapentaenoic acid | 24:5 (n-6) |

Conjugated fatty acids could also be used alone or in mixtures with other fatty acids to create microparticle drug reservoirs that have desired API solubility/affinity and physical properties.

TABLE 5

Conjugated Fatty Acids

| | | |
|---|---|---|
| Common name | Chemical name | Lipid Number |
| Rumenic acid | 9Z,11E-octadeca-9,11-dienoic acid | 18:2 (n-7) |
| - | 10E,12Z-octadeca-10,12-dienoic acid | 18:2 (n-6) |
| α-Calendic acid | 8E,10E,12Z-octadecatrienoic acid | 18:3 (n-6) |
| β-Calendic acid | 8E,10E,12E-octadecatrienoic acid | 18:3 (n-6) |
| Jacaric acid | 8Z,10E,12Z-octadecatrienoic acid | 18:3 (n-6) |
| α-Eleostearic acid | 9Z,11E,13E-octadeca-9,11,13-trienoic acid | 18:3 (n-5) |
| β-Eleostearic acid | 9E,11E,13E-octadeca-9,11,13-trienoic acid | 18:3 (n-5) |
| Catalpic acid | 9Z,11Z,13E-octadeca-9,11,13-trienoic acid | 18:3 (n-5) |
| Punicic acid | 9Z,11E,13Z-octadeca-9,11,13-trienoic acid | 18:3 (n-5) |
| Rumelenic acid | 9E,11Z,15E-octadeca-9,11,15-trienoic acid | 18:3 (n-3) |
| α-Parinaric acid | 9E,11Z,13Z,15E-octadeca-9,11,13,15-tetraenoic acid | 18:4 (n-3) |
| β-Parinaric acid | all trans-octadeca-9,11,13,15-tetraenoic acid | 18:4 (n-3) |
| Bosseopentaenoic acid | 5Z,8Z,10E,12E,14Z-eicosapentaenoic acid | 20:5 (n-6) |

The lipid phase drug reservoir microparticles may also be created from animal ester waxes such as bees wax, vegetable waxes, lanolin, and derivatives. Animal ester waxes typically contain triacontanyl palitate and mixtures of palmitate, palmitoleate, oleate esters, triglycerides and aliphatic alcohols. Additives such as cholesterol, tryglycerides and aliphatic alcohols may be added to change the physical properties of the lipid phase, solubility and affinity of the API to the lipid phase and act as a carrier molecule to help the API diffuse out the lipid phase.

Mineral waxes, mineral oils and lanolin derivatives may be added to change physical and chemical properties of the lipid phase.

Plant sourced waxes can also be used to create the lipid phase. Plant waxes provide an advantage over animal waxes in being easier to control environmental conditions and the same organism (palm or plant) lead to lower batch-to-batch variability. Suitable animal and plant waxes are shown in Table 8. In certain embodiments, the lipid phase is comprised of a carnauba wax. In further embodiments, the fatty acid microparticle is comprised of a combination of carnauba wax and a fatty acid. The addition of fatty acids has the effect of thinning the carnauba in embodiments where a non-solid lipid phase is desirable. In exemplary implementations, the mixture is of carnauba wax and oleic acid, caproic acid, caprylic acid, and/or mixtures of the foregoing.

TABLE 6

Examples of Melting points of fatty acids

| Name | Carbon number | Melting point (°C) |
|---|---|---|
| Capric Acid | 10 | 32 |
| Lauric Acid | 12 | 43 |
| Myristic Acid | 14 | 54 |
| Palmitic Acid | 16 | 62 |
| Stearic Acid | 18 | 69 |
| Arachidic Acid | 20 | 76 |
| Oleic Acid | 18:1 (n-9) | 16 |
| Linoleic Acid | 18:2 | -5 |

TABLE 7

Example of lowering melting temperature of a stearic acid oleic acid mixture

| OA:SA Ratio | Melting Temp °C |
|---|---|
| 0.93 | 32 |
| 0.85 | 37 |
| 0.81 | 45 |
| 0.77 | 42 |
| 0.75 | 47 |
| 0.70 | 51 |
| 0.65 | 48 |
| 0.55 | 57 |
| 0.50 | 56 |
| 0.45 | 59 |
| 0.40 | 60 |
| 0.35 | 63 |
| 0.31 | 64 |

TABLE 2

| Source of Common Animal and Plant Waxes | |
|---|---|
| Name | Source |
| Animal Wax | |
| Beeswax | Insects |
| Lanolin | Sheep |
| Chinese wax | Insects |
| Spermaceti | Sperm Whale |
| Shellac | Insect |
| Plant Wax | |
| Bayberry wax | Bayberry fruit |
| Candelilla wax | Shrubs |
| Carnauba wax | Palm Fronds |
| Castor wax | Castor Bean |
| Esparto wax | Esparto Grass |
| Japan wax | Fruit |
| Jojoba Oil | Seed Simmondsia Chinensis |
| Ouricury wax | Palm Fronds |
| Rice bran wax | Rice Bran |
| Soy wax | Soy Oil |
| Tallow tree wax | Tallow Tree Seeds |

Triglycerides are an alternative to pure fatty acids. They have similar physical properties to the pure counterpart and similar solubility of anesthetics. Triglycerides are better tolerated as they are found throughout the body and there are metabolic pathways to absorb and metabolize the lipid. Table 9 lists triglycerides that can be substituted for fatty acids as a lipid drug reservoir particle. In general, even number fatty acid components are selected because the even number fatty acids are more present in tissues. There are some odd number fatty acid triglycerides that are utilized in the body such as triheptanoin found in milk, which are also suitable. Unsaturated fatty acid based triglycerides such as triolein can be used to soften lipid particles or create emulsion droplets if a multiphase formulation is desired. Unsaturated triglycerides are found throughout the body such as tripalmitolein a main component of mammalian fat. Utilizing triglycerides already found in the body increases tolerability and/or reduces likelihood of adverse reactions. In certain embodiments, the concentration of anesthetic agent within a triglyceride microparticle is from about 1-to about 25% by weight.

TABLE 3

Triglycerides

| | | | |
|---|---|---|---|
| Common Name | Fatty Acid Component | Structure | Fatty Acid Saturation |
| Tripropionin | Propanoic acid | C₁₂H₂₀O₆ | C3:0 |
| Tributyrin | Butyric acid | C₁₅H₂₆O₆ | C4:0 |
| Trivalerin | Valeric acid | C₁₈H₃₂O₆ | C5:0 |
| Tricaproin | Caproic acid | C₁₅H₂₆O₆ | C6:0 |
| Triheptanoin | Heptanoic acid | C₂₄H₄₄O₆ | C7:0 |
| Tricaprylin | Caprylic acid | C₂₇H₅₀O₆ | C8:0 |
| Tripelarigonin | Pelargonic acid | C₃₀H₅₆O₆ | C9:0 |
| Tricaprin | Decanoic acid | C₃₃H₆₂O₆ | C10:0 |
| Triundcylin | Undecanoic acid | C₃₆H₆₈0₆ | C11:0 |
| Trilaurin | Lauric acid | C₃₉H₇₄O₆ | C12:0 |
| Tritridecanoin | Tridecanoic acid | C₄₂H₈₀O₆ | C13:0 |
| Trimyristin | Myristic acid | C₄₅H₈₆O₆ | C14:0 |
| Tripentadecanoin | Pentadecanoic acid | C₄₈H₉₂O₆ | C15:0 |
| Tripalmitin | Palmitic acid | C₅₁H₉₈O₆ | C16:0 |
| Trimargarin | Margaric acid | C₅₄H₁₀₄O₆ | C17:0 |
| Tristearin | Stearic acid | C₅₇H₁₁₀O₆ | C18:0 |
| Triolein | Oleic acid | C₅₇H₁₀₄O₆ | C18:1, cis 9 |
| Trinonadecanoylglycerol | Nonadecanoic acid | C₆₀H₁₁₆O₆ | C19:0 |
| Triarachidin | Arachidic acid | C₆₃H₁₂₂O₆ | C20:0 |
| Triheneicosanoin | Heneicosylic acid | C₆₆H₁₂₈O₆ | C21:0 |
| Trierucin | Erucic Acid | C₆₉H₁₂₈O₆ | C22:cis13, 22:1ω9 |
| Tribehenin | Docosanoic acid | C₆₉H₁₃₄O₆ | C22:0 |
| Tritricosanoin | Tricosanoic acid | C₇₂H₁₄₀O₆ | C23:0 |
| Trilignocerin | Lignoceric acid | C₇₅H₁₄₆O₆ | C24:0 |
| Tripentacosylin | Pentacosylic acid | C₇₈H₁₅₂O₆ | C25:0 |
| Tricerotin | Cerotic acid | C₈₁H₁₅₈O₆ | C26:0 |
| Tricarocerin | Carboceric acid | C₈₄H₁₆₄O₆ | C27:0 |
| Trimontanin | Montanic acid | C₈₇H₁₇₀O₆ | C28:0 |
| Trinonacosylin | Nonacosylic acid | C₉₀H₁₇₆O₆ | C29:0 |
| Trimelissin | Melissic acid | C₉₃H₁₈₂O₆ | C30:0 |
| Trihentriacontylin | Hentriacontylic acid | C₉₆H₁₈₈O₆ | C31:0 |
| Trilacceroin | Lacceroic acid | C₉₉H₁₉₄O₆ | C32:0 |
| Tripsyllin | Psyllic acid | C₁₀₂H₂₀₀O₆ | C33:0 |
| Trigeddin | Geddic acid | C₁₀₅H₂₀₆O₆ | C34:0 |
| Tricerplastin | Ceroplastic acid | C₁₀₈H₂₁₂O₆ | C35:0 |
| Trihexatriacontylin | Hexatriacontylic acid | C₁₁₁H₂₁₈O₆ | C36:0 |
| Triheptatriacontylin | Heptatriacontylic acid | C₁₁₄H₂₂₄O₆ | C37:0 |
| Trioctatriacontylin | Octatriacontylic acid | C ₁₁₇H₂₃₀O₆ | C38:0 |
| Trinonatriacontlyin | Nonatriacontylic acid | C₁₂₀H₂₃₆O₆ | C39:0 |
| Tritetracontylin | Tetracontylic acid | C₁₂₂H₂₄₂O₆ | C40:0 |
| Triisopalmitin | Isopalmitic acid | C₅₁H₉₈O₆ | C16:0 |
| Triisostearin | Isostearic acid | C₅₇H₁₁₀O₆ | C18:0 |
| Trilinolein | Linoleic acid | C₅₇H₉₈O₆ | C18:2n-6 |
| Triheptylundecanoin | Heptylundecanoic acid | C₅₇H₁₁₀O₆ | C18:0 |
| Tripalmitolein | Palmitoleic acid | C₅₁H₉₂O₆ | C16:1-8 |
| Triricinolein | Ricinoleic acid | C₅₇H₁₀₄O₉ | C18:1-9, 11-OH |

In certain embodiments, the disclosed echogenic composition contains a plurality of lipid microparticles with varying characteristics in terms of lipid compositions, size, and/or API concentration. In these implementations, mixtures of lipid microparticles are used to improve the elution rate of the drug and tune the elution to produce a steady first order release from the particles. Adjusting the particle volume to carrier phase volume ratio will extend the release duration of the API.

In exemplary implementations, the lipid microparticle is not a liposome.

In certain embodiments, the lipid microparticle is formulated so as to be solid upon being implanted into a subject (e.g. at a temperature of about 37° C.) In further embodiments, the lipid microparticle is formulated so as to be a liquid upon being implanted into a subject, with the effect being that elution rate from such liquid microparticles would increases relative to a solid microparticle with a similar concentration of anesthetic. In still further embodiments, the composition comprises both of the foregoing microparticles so that some microparticles will remain solid and some will become liquid upon implantation into the subject. The relative balance of the two types of microparticles can be adjusted to achieve the desired elution characteristics.

The size of the lipid microparticle ranges in size from about 1 µm to about 20 µm, in certain implementations. In further embodiments, the lipid microparticle ranges in size from about 5 µm to about 15 µm. In certain exemplary embodiments, the lipid microparticle is about 7 µm.

In certain implementations, elution properties of the disclosed echogenic composition are affected by the volumetric ratio of the aqueous phase to the lipid phase in the composition. According to certain embodiments, the ratio of aqueous to lipid phase is about 50%-80% aqueous phase volume to about 20%-50% lipid phase volume. According to further embodiments, the ratio of aqueous to lipid phase is about 60%-80% aqueous phase volume to about 20%-40% lipid phase volume. According to still further embodiments, the ratio of aqueous to lipid phase is about 70% aqueous phase volume to about 30% lipid phase volume.

According to certain further embodiments, the echogenic composition comprises two or more lipid phases within the aqueous carrier phase. In certain implementations of these embodiments, distributed within the aqueous phase are lipid microparticle phase, as described previously, and a secondary lipid phase which may take to form of an emulsion within the aqueous phase or a plurality of lipid microparticles from which the API elutes at a faster rate than the primary lipid microparticle phase. The purpose of the aqueous phase is to carry the microparticles and secondary lipid phase and keep these components homogenous throughout the formulation. It provides volume so that an accurate dose can be delivered to the desired tissue site and may contain a salt form of the anesthetic, ropivacaine in these examples. The salt form of the anesthetic delivers an upfront burst of drug that matches a similar dose of the saline form of the anesthetic. The primary lipid phase, or drug reservoir microparticle, contains the largest amount of anesthetic in base form and will elute the drug component into the aqueous phase slowly after the upfront burst has eluted from the drug product and into the surrounding tissue. There is a mass transfer limitation due to the solubility of the base form in the aqueous carrier phase and the hydrophilic lipophilic balance (HLB) ratio of the microparticles. The base form has a higher affinity for the lipid phase and the lipid phase will always have some anesthetic present after the elution is complete due to the affinity of the drug for the lipid phase. The secondary lipid phase, or emulsion phase (in some formulations this may be a second type of solid particle), delivers anesthetic at a faster rate than the solid phase microparticles and together they raise the elution rate in the intermediate phase. Once the targeted duration has been met, the elution rate decreases to zero and is below the pharmaceutically effective dose. In certain embodiments, the composition includes and emulsion phase as described above, but without the plurality of solid microparticles.

Suitable lipids for the secondary lipid emulsion phase are any lipid or mixture of lipids that are liquid at 37°. Examples include, but are not limited to stearic acid, oleic acid, caprylic acid, capric acid, lauric acid, palmitic acid, arachidic acid, lignoceric acid, cerotic acid. In certain embodiments, a mixture of stearic acid and oleic acid are the lipids in the lipid emulsion phase. In further embodiments, triglycerides (e.g. trioleate or tripalmitin and trioleate) form the secondary lipid emulsion phase. According to certain embodiments, an emulsifier is used to stabilize the emulsion. Emulsifiers such as TWEEN or other emulsifiers known in the art are suitable.

In certain implementations, anesthetic elution properties of the disclosed composition are affected by the volumetric ratio the two or more lipid phases. According to certain embodiments, the ratio of solid microparticle lipid phase to the emulsion lipid phase is about 50%-75% solid phase volume to about 25%-50% emulsion phase volume. According to certain embodiments, the ratio of solid microparticle lipid phase to the emulsion lipid phase is about 66% solid phase volume to about 34% emulsion phase volume.

### Methods of Formulating Lipid Microparticle Hydrogel Composition

According to certain embodiments, lipid microparticles are generated by agitating a solution of fatty acid phase containing API in a much larger volume of aqueous phase. The preferred ratio of aqueous to lipid phase is 95%-99.5% aqueous phase to 0.5%-5% lipid phase. It is preferred that the aqueous phase be saturated with the API that is present in the lipid phase. In certain embodiments, a salt in >25 mmol concentration is present in the aqueous phase preferably between 25 and 150 mmol, more preferred to be between 45 and 65 mmol. Tyramine substituted hyaluronic acid is present in the aqueous phase at 0.1% to 4% preferably between 0.1 to 1% and specifically at 0.25% concentration. The two-phase mixture is agitated and cooled until microparticles are generated. The particles are concentrated using a centrifuge, filter or settling tank and the aqueous phase decanted leaving the microparticles behind. Additional aqueous phase containing tyramine substituted hyaluronic acid and horse radish peroxidase is added to the free microparticles and the particles are suspended in the solution at a volume ratio of 30% lipid phase to 70% aqueous phase. A hydrogel is formed with the addition of hydrogen peroxide. The hydrogel maintains particle separation and allows for easy delivery via syringe.

According to certain embodiments, formulations with two or more lipid phases (e.g. lipid microparticle and emulsion) the formulation can be prepared as in the preceding paragraph except that prior to the addition of microparticles to the aqueous phase, anesthetic dissolved in a liquid lipid phase (in certain embodiments a mixture of stearic acid and oleic acid) and mixed vigorously with the aqueous phase until an emulsion is formed. Following the formation of the emulation, the lipid microparticles are added as described previously.

Without wishing to bound to theory, it is believed that the zeta potential is increased by adding the salt (e.g., NaCl) to the aqueous phase, causing the surface charge to increase and cause the particles to repel each other allowing smaller diameter particles to form and preventing coalescing particles from forming larger particles prior to solidification. In certain implementations, the hydrogel comprises between 10 mM and about 70 mM salt. In further implementations, salt concentration is between about 25 mM and about 50 mM salt. In further implementations, the hydrogel comprises at least about 50 mM salt. In certain aspects, the salt is NaCl. As will be appreciated by those skilled in the art, other salts are possible.

In certain implementations of the disclosed composition, the anesthetic agent comprises Ropivacaine. In exemplary aspects, the ropivacaine is present in the lipid microparticles in an amount of from about 1 to about 25%. In further embodiments, where the lipid microparticles are comprised of triglycerides,

According to certain alternative embodiments, anesthetic unbound by the plurality of lipid microparticles is dispersed throughout the hydrogel. According to these embodiments, the API dispersed throughout the hydrogel provides for an immediate burst dose, while the API bound in the lipid microparticles provides for extended sustained release.

In certain implementations, the composition further comprises a radiopaque contrast agent.

Further disclosed herein is a method of treating post-operative pain in a subject in need thereof comprising administering to the subject and effective amount of the disclosed echogenic composition. In certain implementations, the immiscible carrier phase is a hydrogel, a viscous liquid, a stable emulsion, or a cream.

In exemplary implementations, the immiscible carrier phase is a hydrogel (e.g., a hydrogel comprised of tyramine substituted hyaluronic acid).

In certain embodiments, the anesthetic is selected from: ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof . In certain implementations, the anesthetic is Ropivacaine. In certain alternative embodiments the anesthetic in bupivacaine.

In certain implementations of the disclosed method, the echogenic composition is administered to the subject and is delivered near a never or nerve bundle of a subject. Ultrasound is then used confirm proximity to the nerve bundle of the subject. In exemplary embodiments, the nerve or nerve bundle innervates the surgical incision area of the subject. The composition may be delivered by way of a syringe or hypodermic needle, other delivery methods known in the art. In exemplary implementations of the disclosed method, the administration of the composition as described herein provides pain relief for about 72 hours or more.

In certain embodiments, the disclosed echogenic composition may comprise a chemotherapeutic agent and the composition is delivered to a site near a tumor. Ultrasound is then used to confirm proximity of the composition to the tumor. In further embodiments, the disclosed methods are used to treat any disease or condition for which being able to determine the localization of API targeted delivery is useful.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

### EXPERIMENTAL

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

In this study, the ultrasonographic characteristics of INSB200 were tested and the results compared the results of commonly used aqueous phase local anesthetic preparations.

### Material and Methods

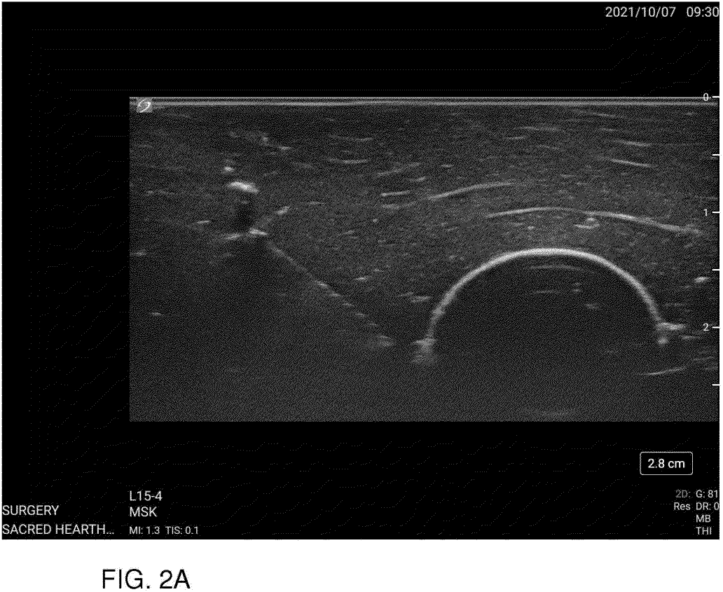

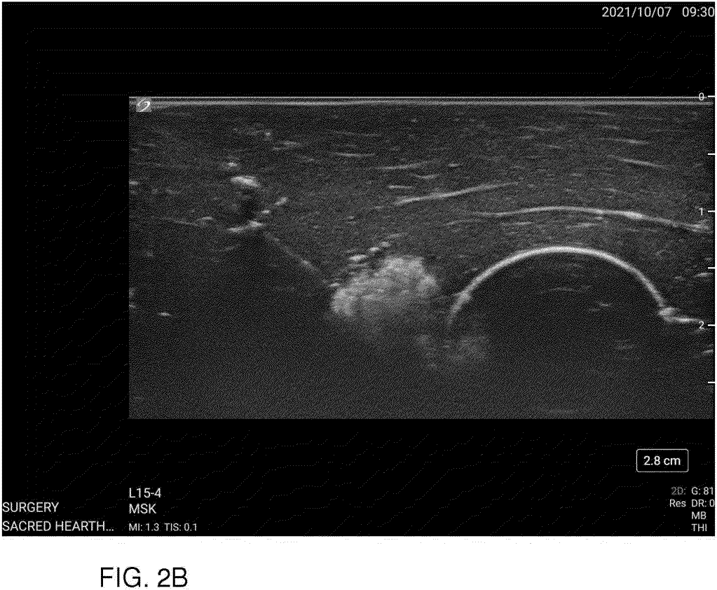

In a room-temperature deceased-porcine (butchered ham and liver) model, we sequentially injected commonly used local anesthetics: Ropivacaine (0.5%), bupivacaine (0.5%), and liposomal bupivacaine (Exparel). In addition, we injected two different preparations of hydrogel-lipid-microparticle matrix ropivacaine (27 mg/mL and 38 mg/mL). The medicant was deposited into the test medium via 21 gauge - 50 mm needle at approximately 2.5 cm depth for each sample and performed under ultrasound guidance (Sonosite PX and 15-4 MHz probe). Pre and post injection images (FIGS. 1A-2B) were collected for each preparation and evaluated for sample echogenicity and tissue extravasation.

### Results

The ultrasound characteristics of commonly used aqueous phase ropivacaine 0.5%, bupivacaine 0.5%, and liposomal bupivacaine preparations were consistent with clinical practice—showing a non-echogenic migration of medicant into tissue planes with significant tissue spread. Conversely, injection of both INSB200 preparations resulted in an echogenic pocket of medicant without significant tissue spread (FIGS. 1B and 2B).

### Example 2

Nine test articles were created to determine the echopacity on a drug product injected into the muscles of a pig. This example models the use of the disclosed drug product as a peripheral nerve block agent or as a sustained release drug product formulation. The impact on echopacity of the particle volume relative to the total volume of the test article, composition of the microparticle phase, depth of injection, and method of formulation preparation were tested.

### Test Article

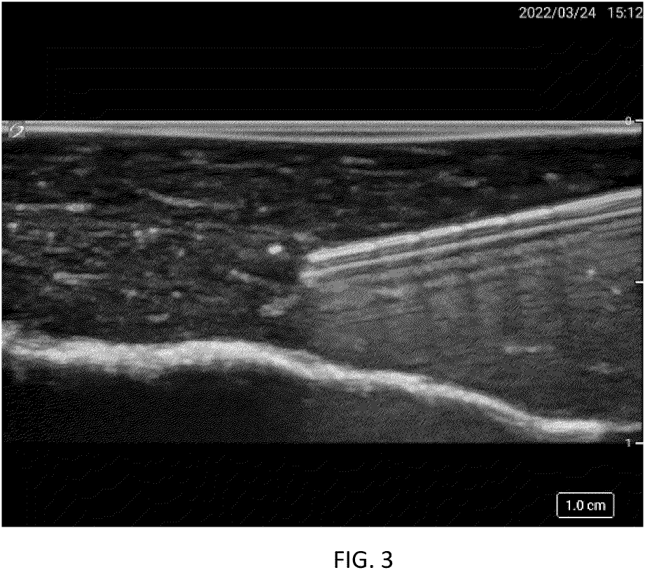

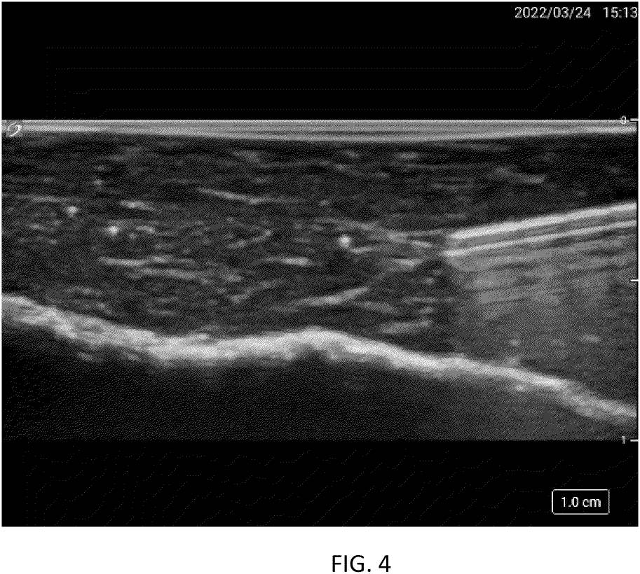

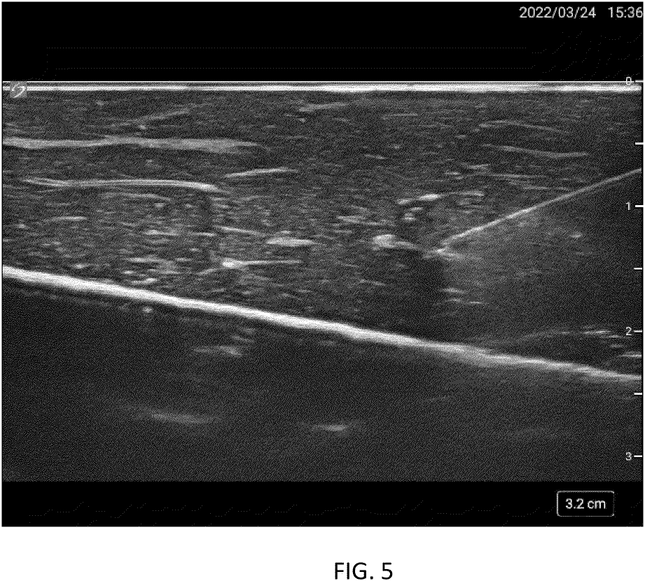

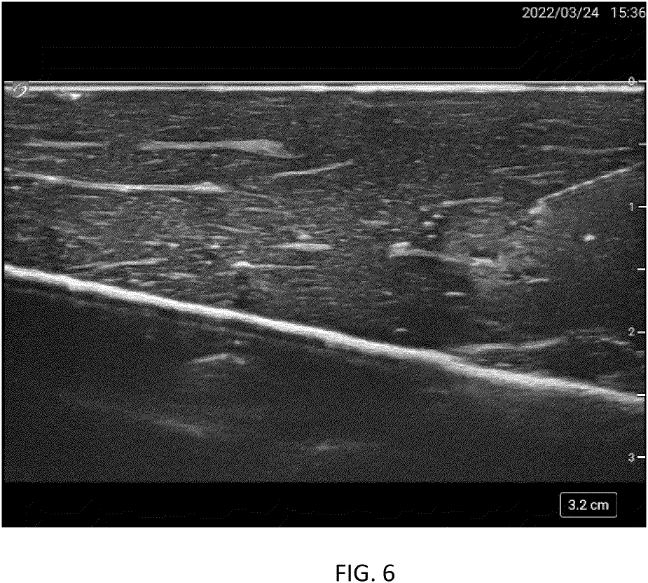

A test article was created by adding 1% by weight hyaluronic acid in a water solution. This solution was injected as a shallow implant and also as a deeper injection. As shown in FIGS. 3-6 the control article has no echopacity relative to the surrounding muscle tissue. In FIG. 2 the injected solution cannot be differentiated from surrounding tissue. In FIG. 4 there is no noticeable differentiation between the control article and the surrounding tissue. The control article is similar to aqueous solutions of analgesic formulations. Injection depth in the muscle did not affect echopacity of the control test article.

### 0.5% Lipid Microparticle

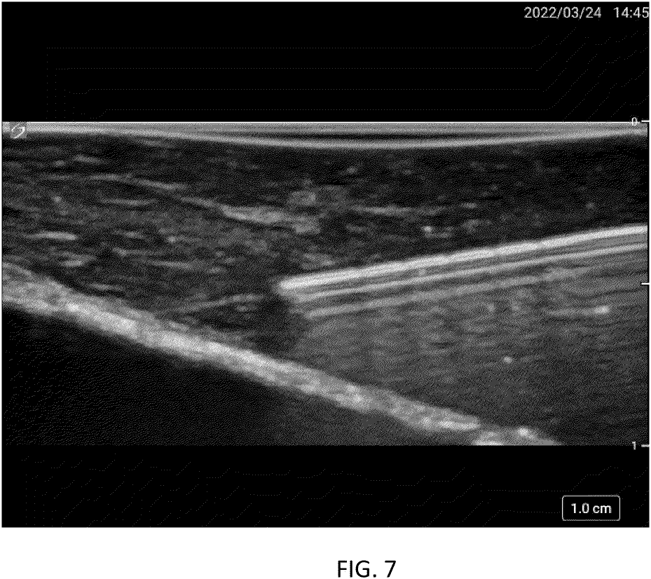

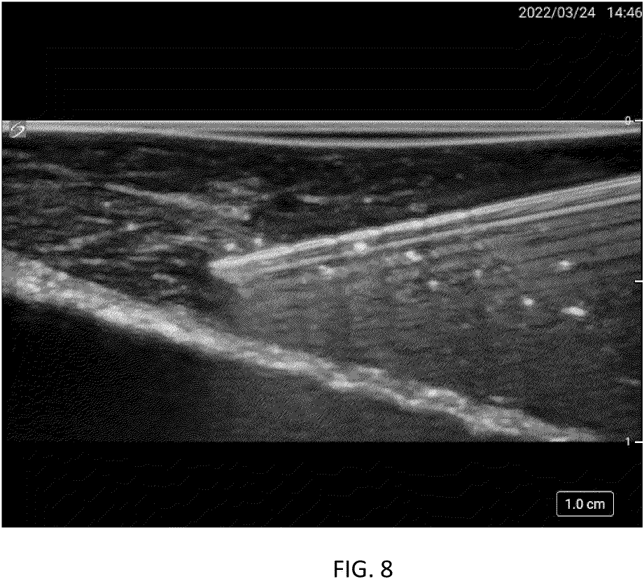

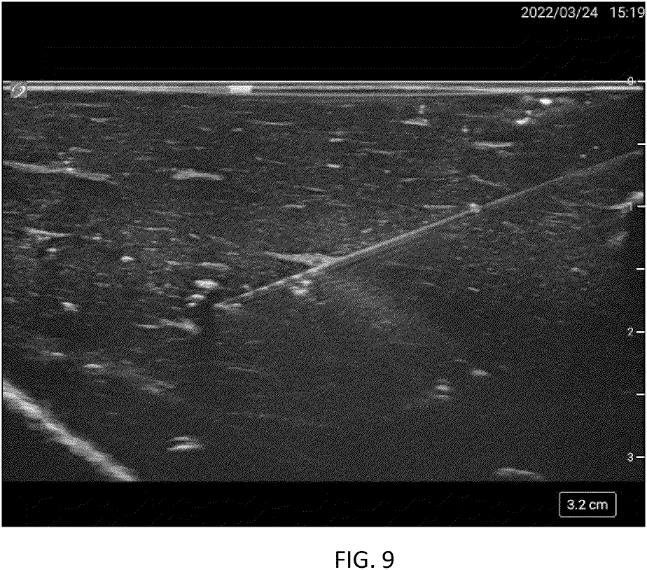

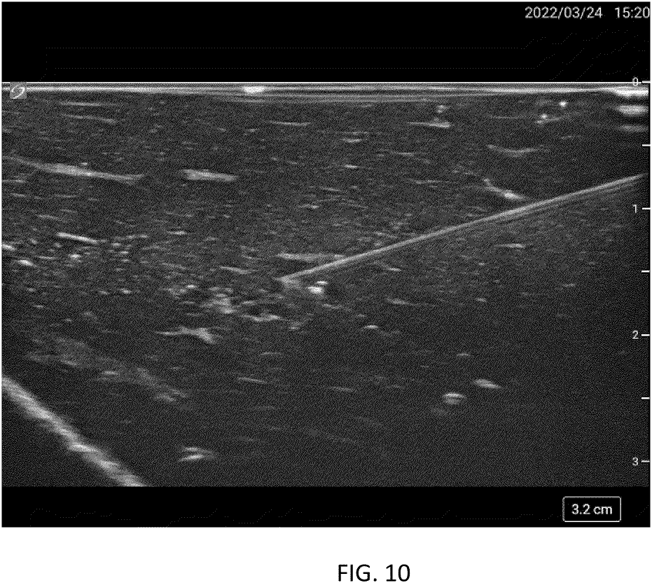

Dilute solutions containing lipid microparticles of biowax (carnauba wax) were prepared by adding 0.5% by volume of a carnauba wax microparticle in a 1% hyaluronic acid solution. Microparticle concentration can be adjusted to adjust the degree of echopacity. Shallow and deep injections were performed to determine if echopacity changes with distance from the probe. The test article did not create an image that could be differentiated from surrounding tissue. FIGS. 7 and 8 show the shallow injections and FIGS. 9 and 10 show the deeper injections. In FIG. 8 the test article showed no echopacity at the needle tip. In FIG. 10 the test article did not produce an image.

### 1.0% Lipid Microparticle

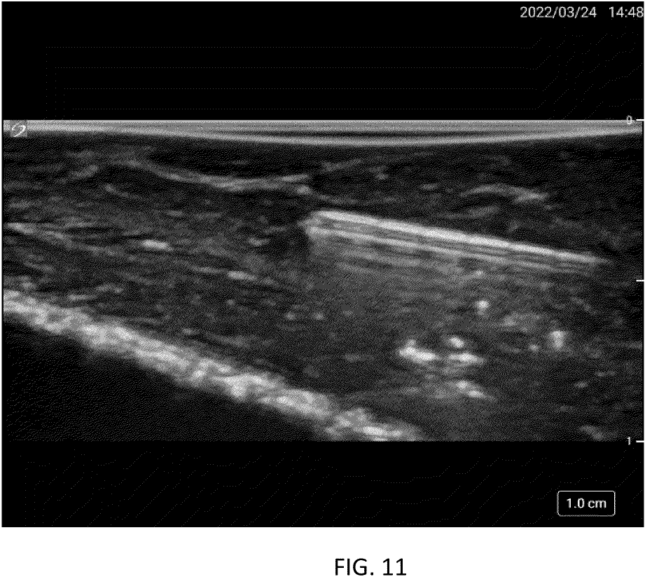

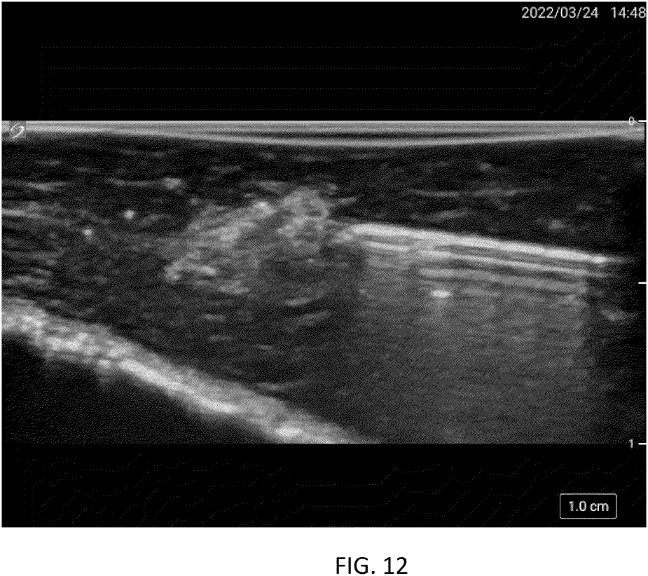

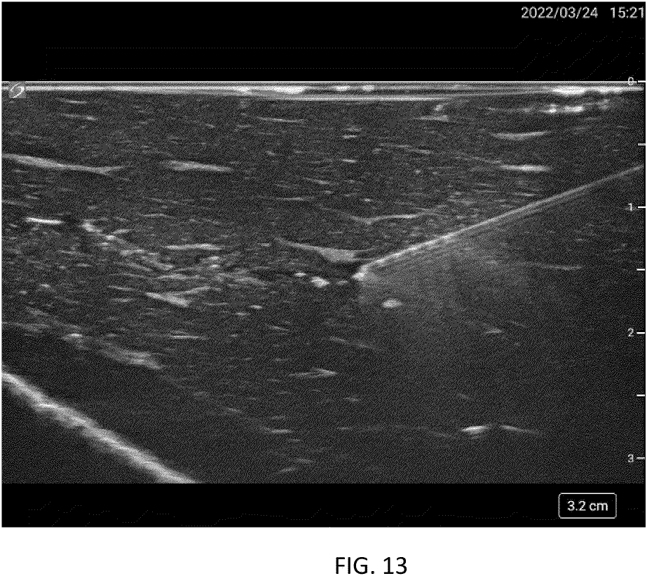

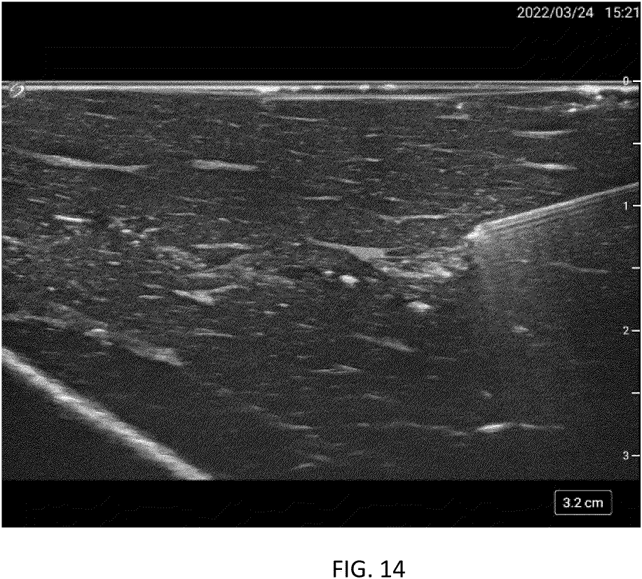

Dilute solutions containing lipid microparticles of biowax (carnauba wax) were prepared by adding 1.0% by volume of a carnauba wax microparticle in a 1% hyaluronic acid solution. Microparticle concentration can be adjusted to adjust the degree of echopacity. Shallow and deep injections were performed to determine if echopacity changes with distance from the probe. The test article created a faint transparent cloud image. FIGS. 11 and 12 show the shallow injections and FIGS. 13 and 14 show the deeper injections. FIG. 12 shows a faint transparent cloud generated at the needle tip. The test article cloud is fainter and less differentiated from surrounding tissue with the deeper injection, shown in FIG. 14. Injection depth does affect echopacity for the 1% lipid microparticle formulation.

### 10% Lipid Microparticle

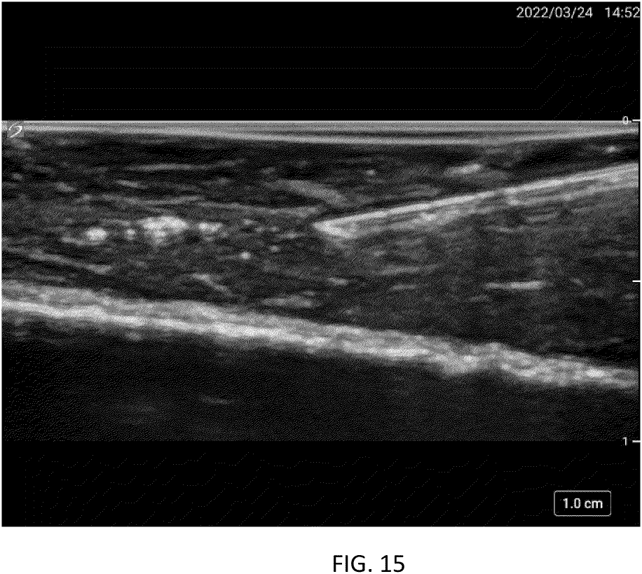

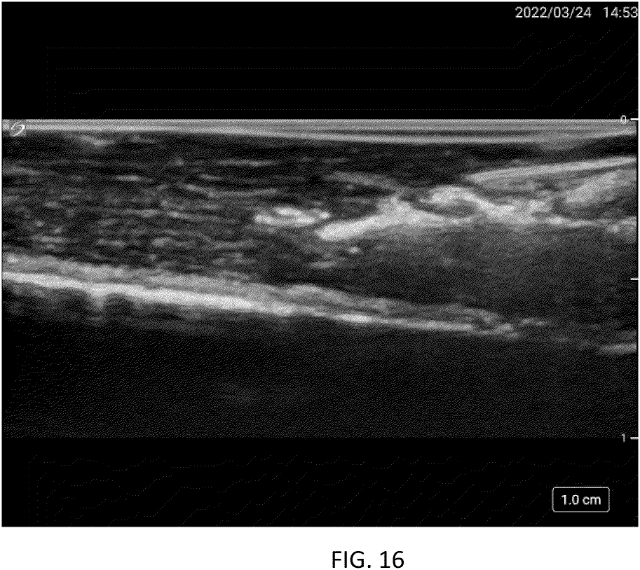

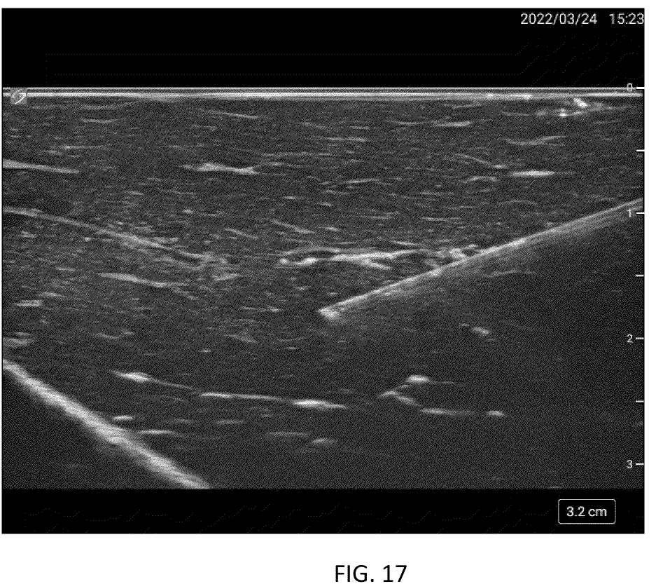

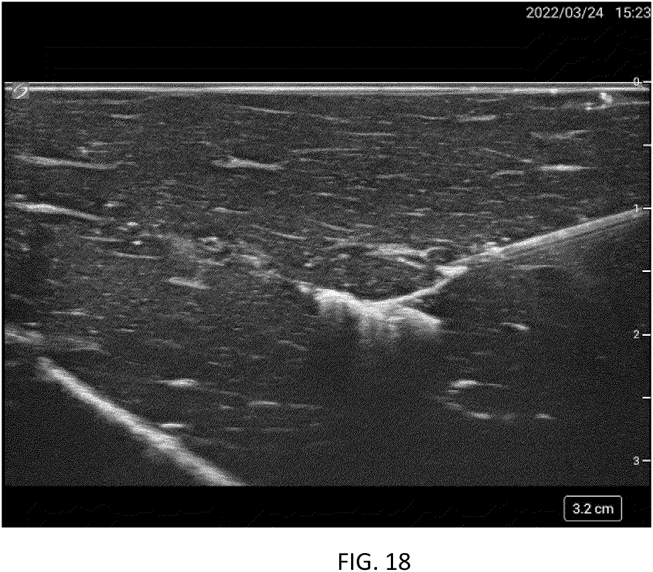

Dilute solutions containing lipid microparticles of biowax (carnauba wax) were prepared by adding 10.0% by volume of a carnauba wax microparticle in a 1% hyaluronic acid solution. The microparticle particle size distribution was 500 nm to about 100 µm in diameter. Microparticle concentration can be adjusted to adjust the degree of echopacity. Shallow and deep injections were performed to determine if echopacity changes with distance from the probe. The test article created a cloud image. FIGS. 15 and 16 show the shallow injections and FIGS. 17 and 18 show the deeper injections. In FIG. 16, the test article cloud begins to obscure and hide the needle. The test article cloud is fainter and less differentiated from surrounding tissue with the deeper injections. In FIG. 18 the test article forms a distinct cloud image significantly brighter than the surrounding tissue. Injection depth does affect echopacity for the 10% lipid microparticle formulation. The test article has a bright proximal surface that creates a shadow distal to the probe.

### 30% Lipid Microparticle

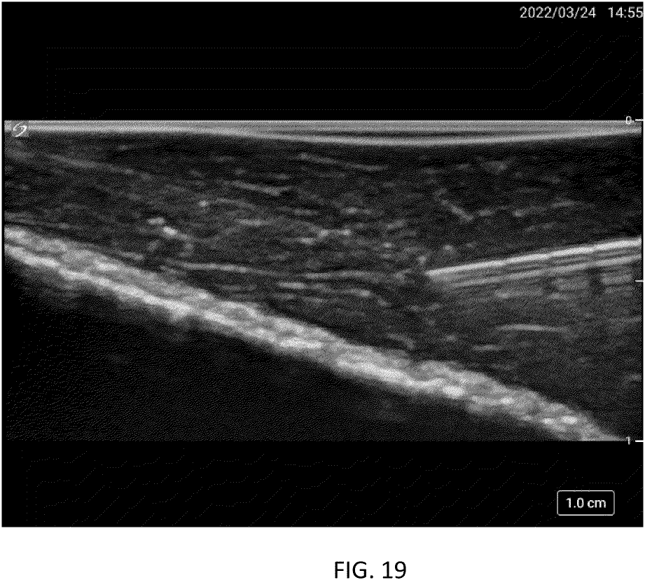

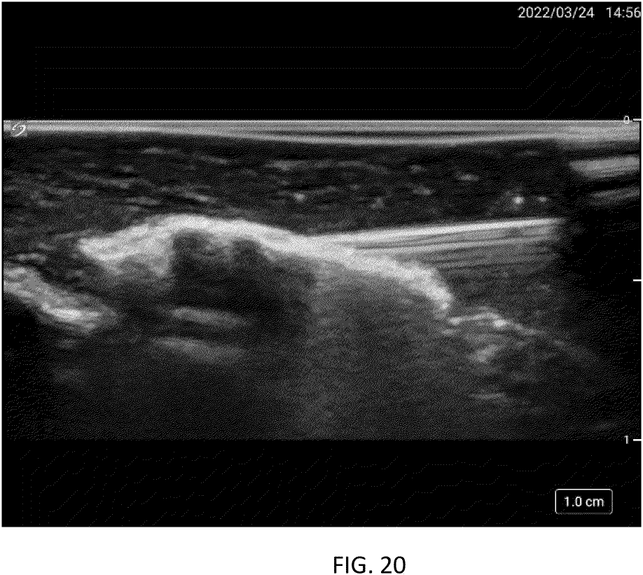

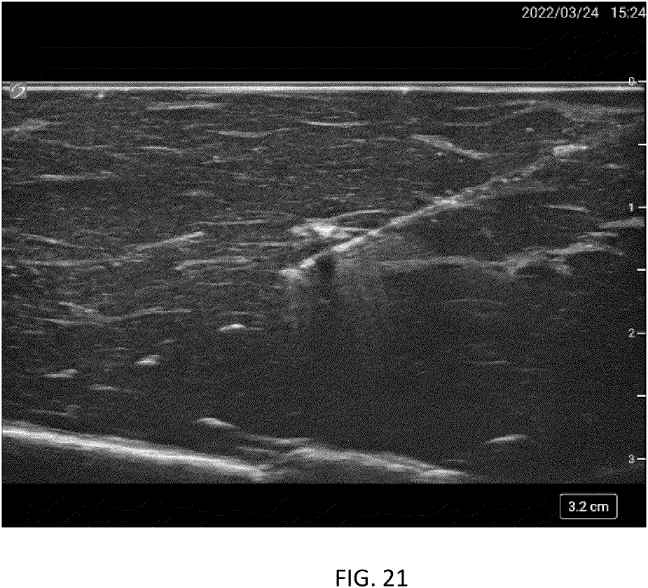

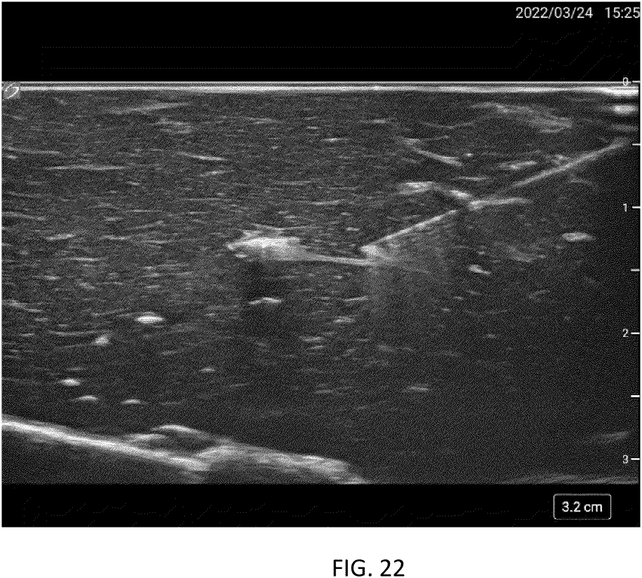

Dilute solutions containing lipid microparticles of biowax (carnauba wax) were prepared by adding 30.0% by volume of a carnauba wax microparticle in a 1% hyaluronic acid solution. The microparticle particle size distribution was 500 nm to about 100 µm in diameter. Microparticle concentration can be adjusted to adjust the degree of echopacity. Shallow and deep injections were performed to determine if echopacity changes with distance from the probe. The test article created a bright cloud image. FIGS. 19 and 20 show the shallow injections and FIGS. 21 and 22 show the deeper injections. In FIG. 20 the test article produced bright top surface reflectance with shadowing and faint reflectance below; muscle fascial plane is obscured below the test article. In FIG. 22 the test article cloud is present in the center of the image, bright reflectance occurs but still allows deeper tissue imaging. The test article cloud is fainter and less differentiated from surrounding tissue with the deeper injection. Injection depth does affect echopacity for the 30% lipid microparticle formulation. The test article has a bright proximal surface that creates a shadow distant to the probe. In the deep injection the distal tissue images were still visible even with a bright reflectance at the test article proximal surface.

### 10% Lipid Microparticle - Homogenized with 13% Ropivacaine API in Lipid Phase

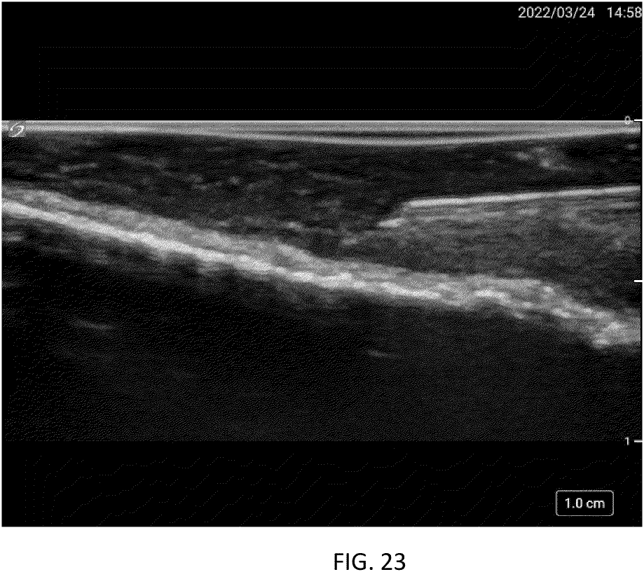

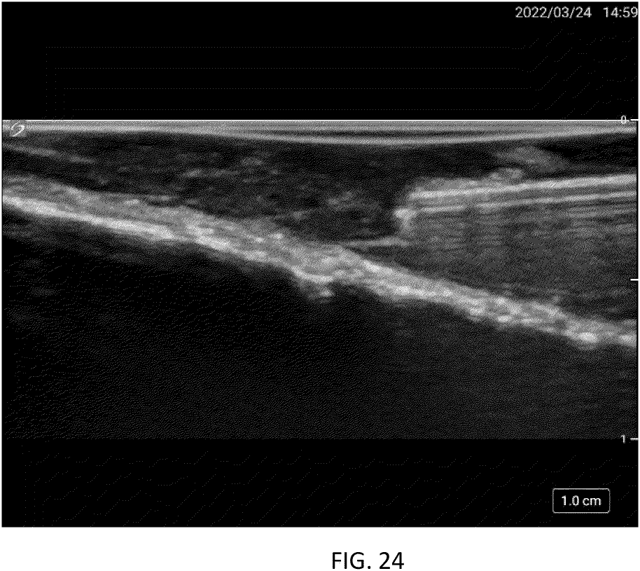

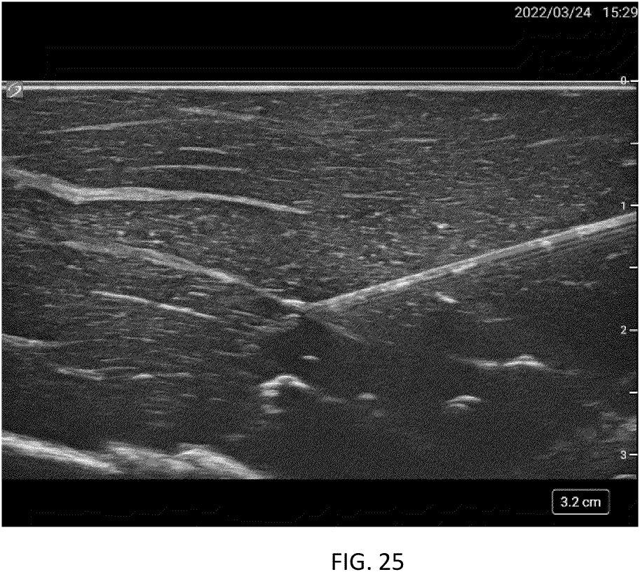

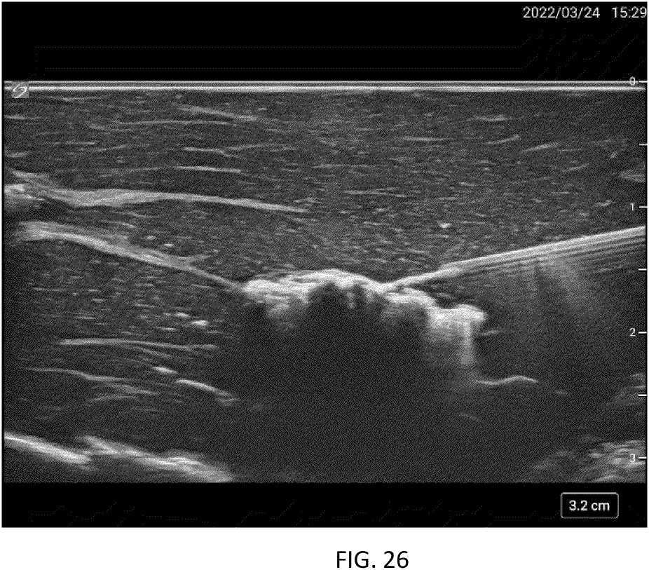

Dilute solutions containing lipid microparticles of biowax (carnauba wax) were prepared by adding 10.0% by volume of a carnauba wax microparticle containing 13% ropivacaine in a 1% hyaluronic acid solution. The samples were homogenized by a rotor - stator homogenizer to break up particle aggregations. The homogenization did not appear to change the particle sizes but did break up aggregated particle clumps. The microparticle particle size distribution was about 500 nm to about 100 µm in diameter. Microparticle concentration can be adjusted to adjust the degree of echopacity. Shallow and deep injections were performed to determine if echopacity changes with distance from the probe. The test article created a bright cloud image. FIGS. 23 and 24 show the shallow injections and FIGS. 25 and 26 show the deeper injections. In FIG. 24 the test article is seen on top of the needle and tip; the test article is less visible in shallow depth with this formulation. In FIG. 26 the test article produces a cloud with bright high reflectance surface and a track to the needle tip, as shown in the center of the image. The test article clouds are bright with higher reflectance. Injection depth does not appear to affect echopacity for the 10% lipid microparticle homogenized formulation. The test article has a bright proximal surface that creates a shadow distant to the probe. In the deep injection the distal tissue images were still visible even with a bright reflectance at the test article proximal surface. The better distribution of the small particles and addition of solid phase API crystals in the lipid phase create a more echogenic drug product.

### 30% Lipid Microparticle Containing Stearic Acid and Tributyrate

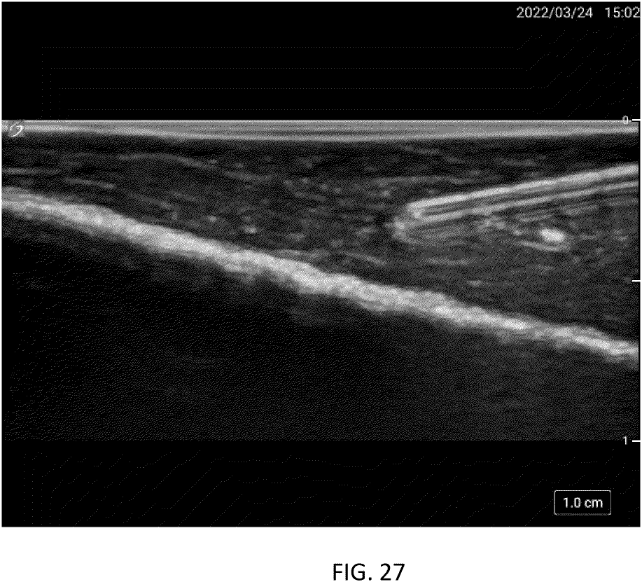

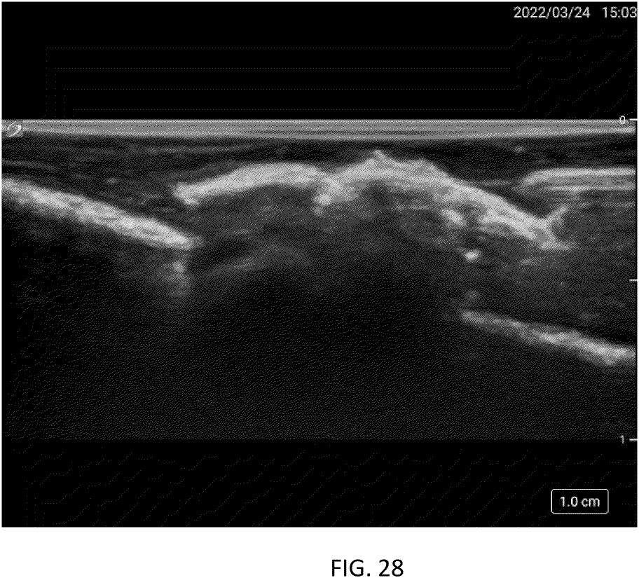

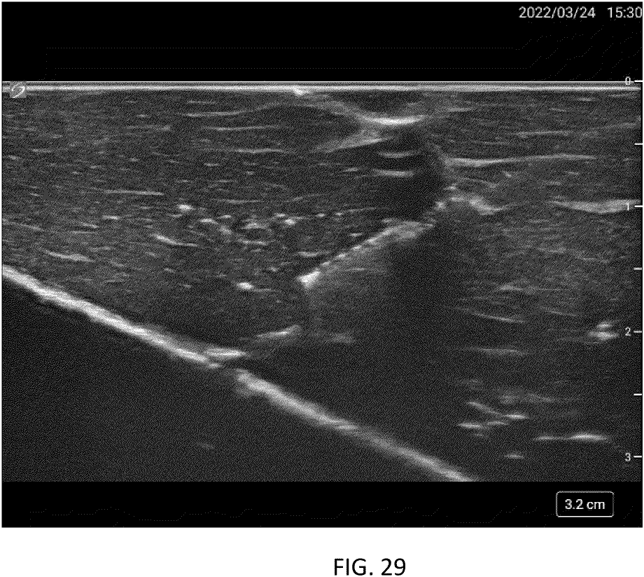

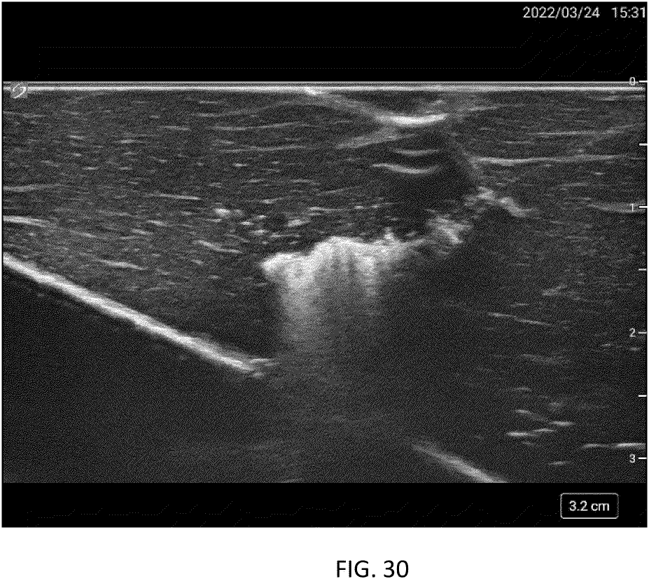

Dilute solutions containing lipid microparticles of fatty acids and triglycerides were prepared by adding 30.0% by volume of a stearic acid/tributyrate (10% tributyrate) microparticle in a 1% hyaluronic acid carrier solution. The microparticle particle size distribution was about 500 nm to about 100 µm in diameter. Microparticle concentration can be changed to adjust the degree of echopacity. Shallow and deep injections were performed to determine if echopacity changes with distance from the probe. The test article created a bright cloud image. FIGS. 27 and 28 show the shallow injections and FIGS. 29 and 30 show the deeper injections. In FIG. 28 the test article produced a bright reflective surface that obscures the tissue feature distal to the probe, with shallow injections. FIG. 30 shows a test article that produced a bright reflectance on the proximal side and occludes the tissue features below the test article. The test article cloud is bright and clearly visible. Injection depth does affect echopacity for the 30% lipid microparticle formulation. The test article has a bright proximal surface that creates a shadow distant to the probe. In the deep injection the distal tissue images were still visible even with a bright reflectance at the test article proximal surface.

### 10% Lipid Microparticle Containing Stearic Acid and Tributyrate

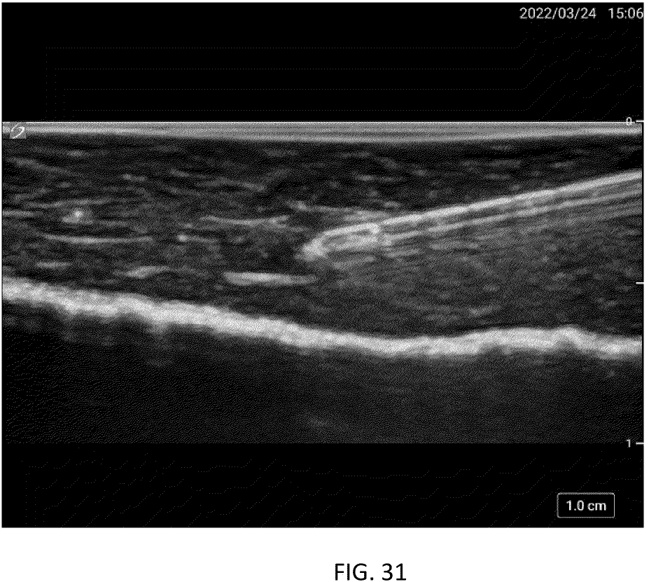

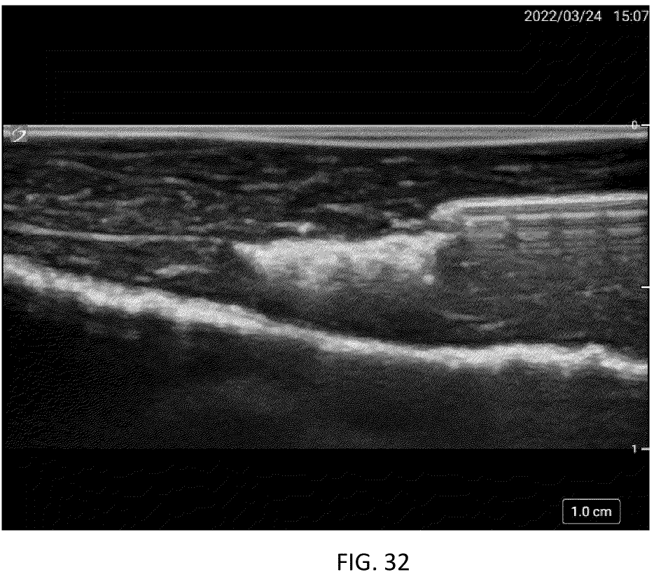

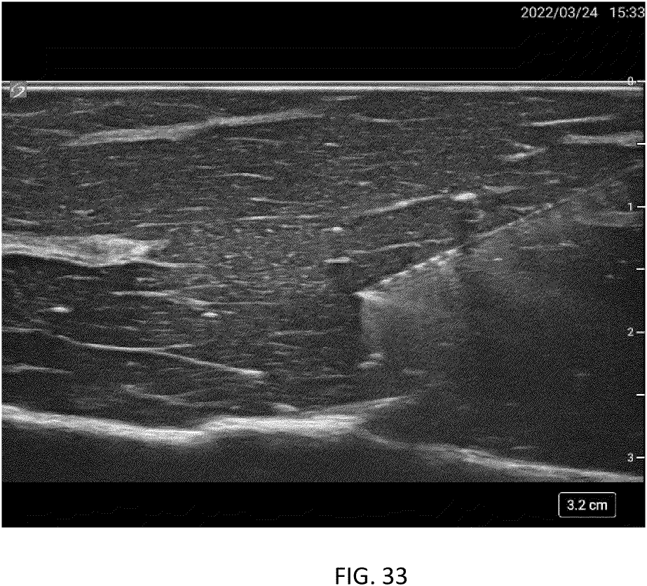

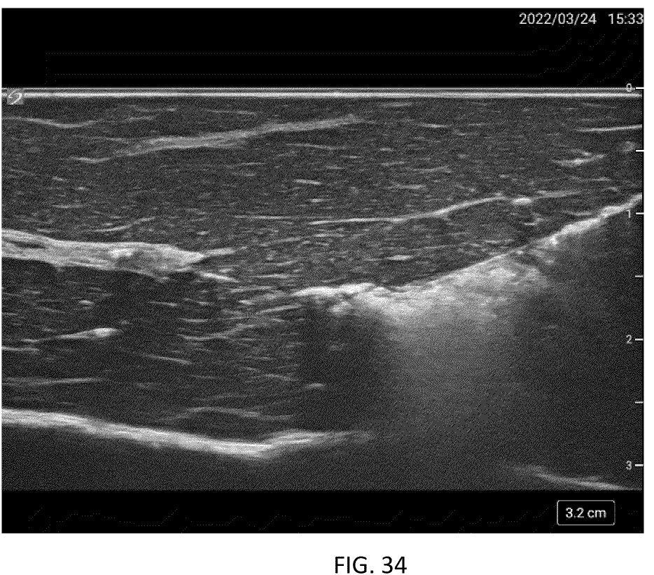

FIGS. 31 and 32 show the shallow injections and FIGS. 33 and 34 show the deeper injections. In FIG. 32 the test article is clearly visible in the center of the field of view. In FIG. 34 the test article could obscured the tissue features below. 10% loading of microparticles produces a bright visible cloud in the shallow injection shown in FIG. 32. FIG. 31 shows the tissue prior to injection. FIG. 33 shows tissue prior to injection and the needle can be seen entering the right side of the image. FIG. 34 shows the test article produces a bright cloud at deep injection. 10% lipid microparticle loading does produce desired visibility of the test article.

### 10% Lipid Microparticle Containing Caprylic Acid and Tristearate

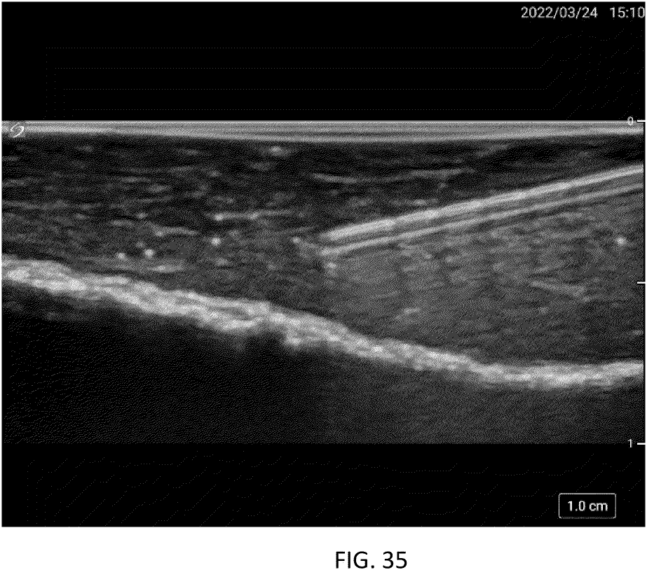

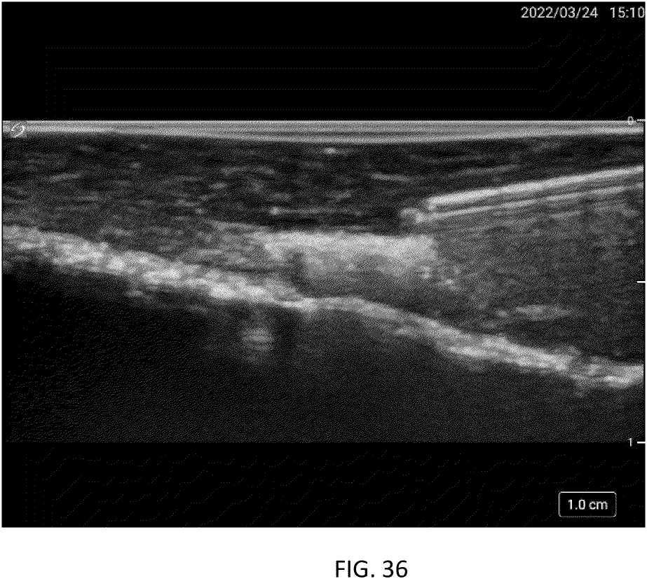

FIGS. 35 and 36 show the shallow injection. The test article can be seen at the tip of the needle. Changing the composition to caprylic acid in a tristearate base still produces a bright image. Solid phase particles produce a bright image. Test article cloud seen below needle tip. In FIG. 36, tissue features can be seen below test articles. The formulation does not occlude tissue features below the implant.

### Example 3

### New Formulation and Echogenic Data

In this Example a 3-phase emulsion formulation was prepared and found to provide excellent sustained release performance for small molecules like ropivacaine but with the advantages of not having a residual drug product left in the body after 7 days. For nerve tissue, this is an advantage since it will not cause irritation or compression of the nerve or surrounding tissue during movement of neighboring muscles. The following is an example of one of the 3-phase formulations.

### INSB200 Manufacturing, Components, and Specifications 30 mL Sample Manufacturing

Sterile filter soybean oil using 0.2 µm vacuum filter. Sterile filter water using 0.2 µm vacuum filter. Create a 0.15% sodium hyaluronate solution by weight, combine the following in a clean glass beaker: (a) 100 mL sterile water and (b) 0.15 g sodium hyaluronate. Seal glass beaker and allow the sodium hyaluronate solution to sit overnight in the fridge (2-8° C.). Add 11 g of filter sterilized soybean oil to 40 mL of acetone mix until the liquids are homogenous. Add 0.75 g of lecithin to the acetone/soy oil mixture. Add 1.43 g of ropivacaine base to the acetone and soy oil mix. The liquid was mixed until all of the ropivacaine had been dissolved in the liquid phase. The resulting liquid was sterile filtered through a 0.2 micron filter to sterilize the solution and transferred into a sterile vessel with a sterilized mixer and condenser connected to a vacuum. Moderate vacuum was applied to the vessel and the liquid bought to a boil while mixing vigorously. Crystals began to form. The crystallization of the ropivacaine in the soy oil phase was continued until there was no more acetone condensing in the condenser. Sterile nitrogen gas was bubbled through the soy oil and ropivacaine crystal slurry to strip the acetone from the soy oil until no more acetone is removed. 0.51 g of glycerol was added to the hyaluronic acid solution and mixed until the liquid was homogenous. The solution was sterile filtered the solution and transferred to the vessel containing the soy oil, lecithin and ropivacaine base solution. The two phases in the main mixing vessel were homogenized until there is a stable emulsion containing ropivacaine base crystals.

Formulation Components:
Soybean Oil: 40% v/v
Glycerol: 1.7% w/vₜₒₜₐₗ
Lecithin from Soybean: 2.5% w/vₜₒₜₐₗ
Ropivacaine Base: 130 mg/g_{soybean} oil
0.15% Sodium Hyaluronate Solution (pH=8): -60% v/v, fill to total volume
Soybean Oil (40% v/v)
Product Name: Soybean oil
CAS-No: 8001-22-7
Glycerol (1.7% w/vₜₒₜₐₗ)
Product Name: Glycerol
CAS No: 56-81-5
Lecithin (2.5% w/vₜₒₜₐₗ)
Product Name: Lecithin (Vegetable/Laboratory)
CAS No: 8002-43-5
0.15% Sodium Hyaluronate Solution (~60% v/v)
Product Description: Sodium hyaluronate
CAS-No: 9067-32-7
Ropivacaine (130 m/g_{soybean}ₒᵢₗ)

CAS-No: 84057-95-4

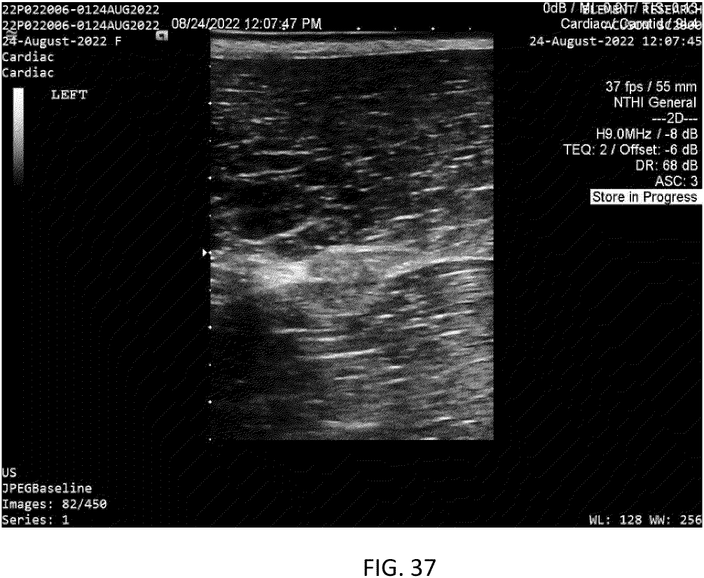

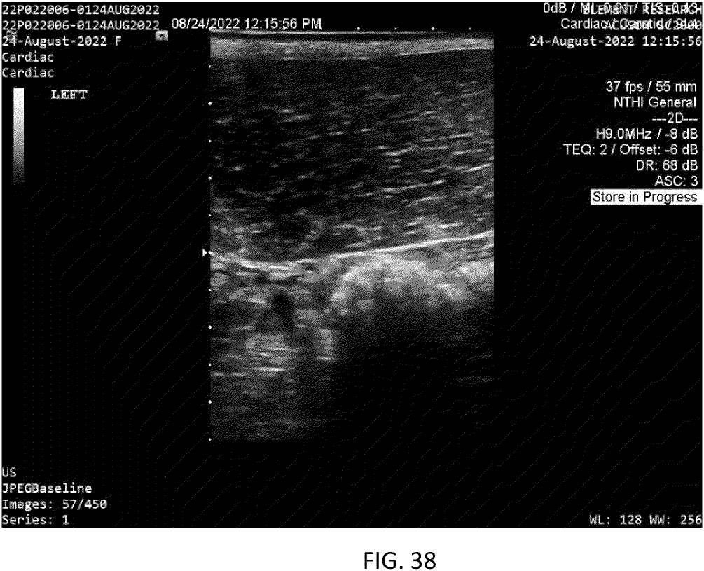

The resulting solution was injected into a pig hind leg in a manner to produce a peripheral nerve block at the sciatic nerve. The pigs were observed to have a full motor block for 48 hrs at the 130 mg/g soy oil dose and fully recovered within 3 days. Necropsy showed that the drug product was completely absent from the injection site. The formulation was found to be echogenic and was an improved way to show that the target nerve was dosed correctly to produce the desired motor and pain block. FIG. 37 shows the pig sciatic nerve prior to injection. FIG. 38 shows the sciatic nerve completely covered by the drug product after injection. Injection was started distally to the ultrasound probe and finished proximal to the probe on subsequent injections. In FIG. 38 the drug product is shown within the intrafascial space below the bright line midway in the field of view. Physicians can now visualize the drug product and ensure that they are targeting the correct nerve. A dose of 20 mL was injected into the pig and normalized to a 30 mL dose for a 70 Kg human.

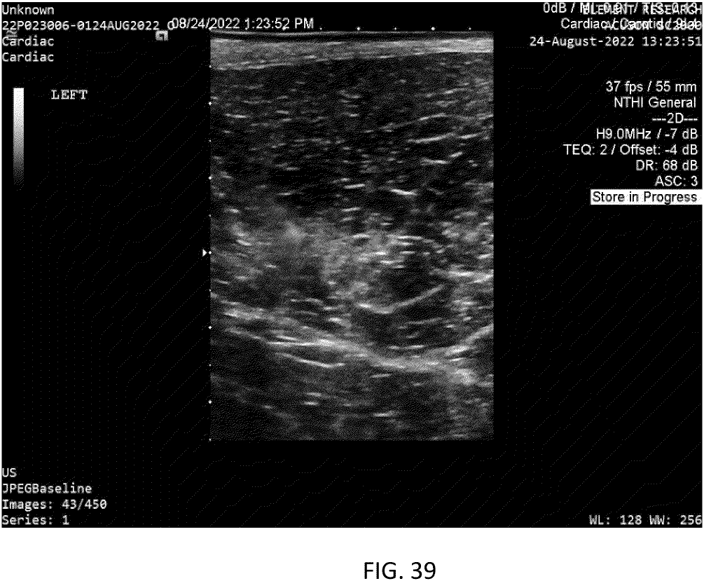

FIG. 39 shows a second pig’s sciatic nerve prior to injection.

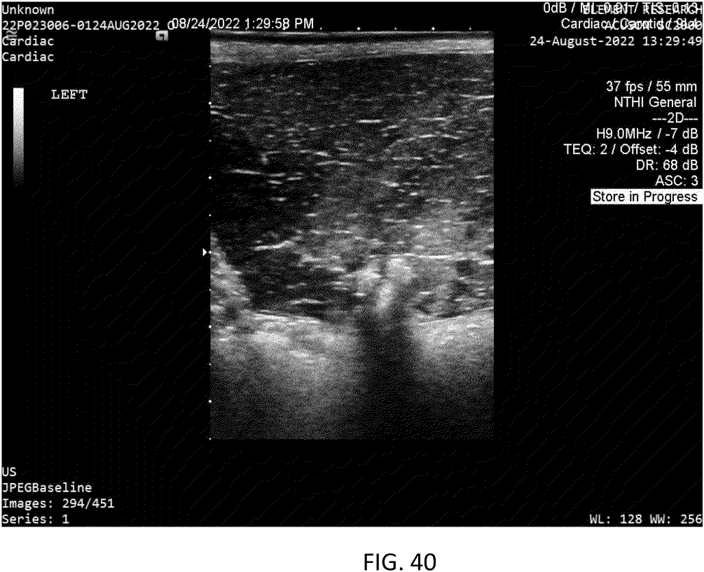

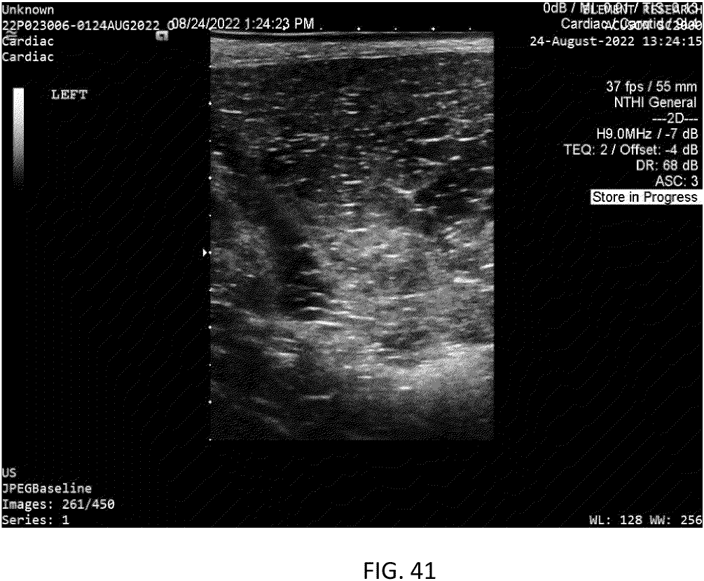

FIGS. 40 and 41 show mid injection ( FIG. 42) and final placement of drug product in the fascial plane next to and enveloping the sciatic nerve (FIG. 41).

An echogenic formulation that comprises a 3-phase stabile emulsion can be injected through a 4 inch long 21 G needle attached to an 18-inch long luer lock pig tail tubing using a 20 mL syringe. The formulation may consist of 0-1.0% hyaluronic acid (sodium hyaluronate, hyaluronan); 0-2.25% glycerol; 0-2.5% soy or egg lecithin; 0-60% soy oil; 0-30% mixed triglycerides; 40-280 mg ropivacaine base/g soy oil; and water.

What is claimed is:

1. A method for treating pain in a subject comprising: administering an echogenic composition to a target site in a subject, the echogenic composition comprising: an aqueous carrier; and
a plurality of lipid microparticles dispersed within the aqueous carrier, wherein the plurality of lipid microparticles comprise and anesthetic agent; and
confirming delivery of the echogenic composition to the target site with ultrasound.

2. The method of claim 1 wherein the aqueous carrier is hydrogel comprised of tyramine substituted hyaluronic acid, wherein the hydrogel is formed through di-tyramine crosslinking and wherein the degree of tyramine substitution of hyaluronic acid hydroxyl groups is about 0.5% to about 3%.

3. The method of claim 1, wherein the volumetric ratio between the aqueous carrier and the lipid microparticles is from about 60-90 the aqueous carrier to about 40-10 lipid microparticles.

4. The method of claim 1, wherein the plurality of lipid microparticles are comprised of a lauric acid and a fatty acid having a carbon number greater than lauric acid.

5. The method of claim 4, wherein the lipid microparticle is a wax and the wax is a carnauba wax.

6. The method of claim 4, wherein the lipid microparticles comprise a wax or a mixture of a wax and a fatty acid, wherein the fatty acid is C4 or greater.

7. The method of claim 6, wherein the lipid microparticle comprises stearic acid and tributyrate.

8. The method of claim 7, wherein the stearic acid and tributyrate are present at a ratio of from about 0.1% to about 30%.

9. The method of claim 1, wherein the anesthetic agent is present within the lipid microparticle in a crystalline form.

10. A method for treating pain in a subject comprising: administering an echogenic composition to a target site in a subject, the echogenic composition comprising: an aqueous carrier; and
lipid phase dispersed into droplets within the aqueous carrier, an undissolved crystalline anesthetic agent within the lipid phase; and
confirming delivery of the echogenic composition to the target site with ultrasound.

11. The method of claim 10, wherein the lipid phase is a triglyceride.

12. The method of claim 11, wherein the triglyceride is a liquid at 25° C.

13. The method of claim 10, wherein the lipid phase droplets are from about 500 nm to about 5 µm in diameter.

14. The method of claim 10, wherein the aqueous phase comprises hyaluronic acid present in an amount from about 0.1% to about 1%.

15. An echogenic composition for the treatment of pain comprising:
a continuous aqueous phase comprising an emulsifier and a polyol;
a lipid phase comprising a triglyceride, wherein the triglyceride is liquid at 25° C. and wherein an undissolved crystalline anesthetic agent within the triglyceride; and wherein the lipid phase is emulsified within the continuous aqueous phase.

16. The composition of claim 15, wherein the emulsifier is hyaluronic acid present in an amount from about 0.15% to about 1%.

17. The composition of claim 15, wherein the lipid phase further comprises a phospholipid present in an amount from about 0.1% to about 2.0% of the lipid phase.

18. The composition of claim 15, wherein the lipid phase further comprises an antioxidant present in an amount of from about 0.01% to about 1% (w/v) of the composition.

19. The composition of the claim 15, wherein the lipid phase is from about 10% to about 40% (w/v).

20. The composition of claim 15, wherein the polyol is glycerol and is present in an amount of from about 0.25 to about 2.5% (w/v) of the composition.
